(19) United States
(12) Patent Application Publication
(10) Pub. No.: US 2018/0271869 A1
(43) Pub. Date: Sep. 27, 2018

(54) TREATMENT OF ANXIETY DISORDERS AND AUTISM SPECTRUM DISORDERS

(71) Applicants: Jie LIU, West Chester, PA (US); Todd A. VERDOORN, Plymouth, MN (US); Gideon SHAPIRO, Gainesville, FL (US); Gang CHEN, Suzhou, Jiangsu Province (CN); Rugen Holdings (Cayman) Limited, Grand Cayman (KY)

(72) Inventors: Jie Liu, West Chester, PA (US); Todd A. Verdoorn, Plymouth, MN (US); Gideon Shapiro, Gainesville, FL (US); Gang Chen, Suzhou, Jiangsu Province (CN)

(21) Appl. No.: 15/513,112

(22) PCT Filed: Sep. 22, 2015
(86) PCT No.: PCT/US2015/051488

§ 371 Date: Mar. 21, 2017

Related U.S. Application Data
(60) Provisional application No. 62/053,645, filed on Sep. 22, 2014.

(51) Int. Cl. A61K 31/519; A61K 31/506; A61K 31/451; A61P 25/22; A61K 45/06; A61K 9/00

(52) U.S. Cl. CPC A61K 31/519; A61K 31/506; A61K 31/451; A61K 9/0085; A61K 45/06; A61K 9/0019; A61P 25/22

(57) ABSTRACT

Disclosed are methods for treating autism spectrum disorders and/or anxiety disorders by administering certain NR2B subunit-selective NMDA (N methyl-D aspartate) antagonists. Anxiety disorders include agoraphobia (with or without panic disorder), generalized anxiety disorder (GAD), social anxiety disorder (SAD), panic disorder (PD), post-traumatic stress disorder (PTSD) and obsessive-compulsive disorder (OCD).

### BACKGROUND

Autism spectrum disorders (ASDs) refer to a series of pervasive developmental disorders that cause severe and ubiquitous impairment in thinking, feeling, language, and the ability to relate to others. The onset is generally before the age of 3 years, and an ASD is usually first diagnosed in early childhood. ASDs can range from a severe form, called autistic disorder, through pervasive development disorder not otherwise specified (PDD-NOS), to a much milder form, commonly known as Asperger's syndrome. ASDs have a prevalence of 0.6% in the population, affecting many more boys than girls (see, Bertrand et al., Pediatrics 2001; 108:1155-61, Yeargin et al., JAMA 2003; 289:49-55, and Newschaffer et al., Pediatrics 2005; 115:e277-82). Twin and family studies have estimated the heritability of autism as being up to 90%, making it one of the most heritable complex disorders. Rare genetic syndromes and known chromosomal anomalies explain roughly 10% of cases of autism, including Fragile X, tuberous sclerosis, Smith-Lemli-Opitz syndrome, and maternally-inherited duplications of the Prader-Willi/Angelman syndrome region (15qII-13). However, despite high heritability, genetic studies to date have not provided substantial insight into the 90% of autism spectrum disorders with idiopathic etiology.

Current therapeutic strategies for the diverse forms of autistic syndromes are mainly targeted to the correction of the most severe behavioral problems or symptoms associated with the main disorder. A number of psychopharmacological agents have been utilized in children with autism, but only to address specific psychiatric or behavioral symptoms, and with mixed results. These agents include: serotonin-related drugs, dopamine-related agents, epinephrine and norepinephrine-related compounds, and a variety of other agents such as opiate antagonists, ACTH, clozapine, risperidone, vitamins B6 and B12 and melatonin. Thus, while children with ASDs are prescribed a number of drugs, there is still no accepted rational therapeutic paradigm designed to address autism or its causes.

Anxiety disorders include a variety of psychological disorders that involve excess fear, worry, avoidance, and compulsive behaviors. Anxiety disorders are among the most common psychiatric diseases in the United States and are responsible for significant morbidity, functional impairment and excessive use of healthcare services. Anxiety disorders have been formally classified into specific conditions including agoraphobia (with or without panic disorder), generalized anxiety disorder (GAD), social anxiety disorder (SAD), panic disorder (PD), post-traumatic stress disorder (PTSD) and obsessive-compulsive disorder (OCD). Patients may suffer from a single specific disorder or a combination of them.

Treatment of anxiety disorders currently relies on drugs that modulate the GABA and/or serotonin receptor systems. Benzodiazepines are a class of drug that increase the tone of inhibitory GABA receptors and have profound anxiolytic effects in humans. Benzodiazepines are most often used in cases of GAD but also have found use in the other forms of anxiety disorders including PD, SAD and PTSD. Selective serotonin reuptake inhibitors (SSSIs) have been widely prescribed as stand-alone or adjunct therapy for all of the anxiety disorders, particularly PD, agoraphobia, social phobia and OCD. Buspirone, a selective 5-HT_{1A} partial agonist has been approved by the United States Food and Drug Administration as an anxiolytic agent and is often used in the treatment of GAD in combination with SSR1s.

Numerous early studies involving serotonin-specific reuptake inhibitors led to the hypothesis that OCD may be associated with dysregulation of serotonergic neurotransmission. Indeed, SSRIs have been established as the first-line pharmacotherapy of OCD. However, approximately one-half of the patients who receive an adequate trial with these agents remain clinically unchanged. In addition SSRIs can have undesirable side effects, such as nausea; nervousness, agitation or restlessness; dizziness; erectile dysfunction or reduced sexual desire; drowsiness; insomnia; weight gain or loss; headache; dry mouth; vomiting; diarrhea; indigestion; blurred vision; excessive sweating Dopamine antagonists have similarly been used to augment treatment of patients resistant to serotonin uptake inhibitors, but these agents have drawbacks in terms of side effects, such as acute dystonias, akathisia, parkinsonian symptoms, and neuroleptic malignant syndrome.

No single drug or protocol has been proven to be entirely satisfactory for addressing the behavioral and psychological symptoms associated with ASDs and/or anxiety disorders as chronically administered therapies. In addition, currently used drugs display limiting side effects. While benzodiazepine drugs can be effective, side effects that circumscribe their chronic use include sedation, memory loss and addiction. SSRIs are amenable to chronic use but still have a high incidence of side effects that limit quality of life, such as impeded sexual function.

### BRIEF SUMMARY

There is a continuing need in the art for compounds that can address the underlying causes of autism spectrum disorders and/or anxiety disorders and alleviate the associated behavioral abnormalities common to these conditions. The present invention encompasses the insight that certain NR2B subunit selective NMDA receptor antagonists are effective for the treatment of autism spectrum disorders and/or anxiety disorders.

In some aspects, the present invention provides a method for treating an autism spectrum disorder (ASD) in an individual in need thereof comprising administering to the individual an effective amount of a certain NR2B subunit selective N-Methyl-D-aspartate (NMDA) inhibitor.

In some aspects, the present invention provides a method for treating an anxiety disorder in an individual in need thereof comprising administering to the individual an effective amount of a certain NR2B subunit selective N-Methyl-D-aspartate (NMDA) inhibitor.

In some embodiments, the NR2B subunit selective NMDA inhibitor is traxoprodil (CP-101,606), which is (+)-(1S,2S)-1-(4-hydroxyphenyl)-2-(4-hydroxy-4-phenylpiperidino)-1-propanol (“Compound A”):

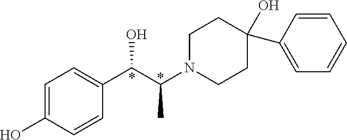 [SMILES: C[C@@H]([C@@H](O)c1ccc(O)cc1)N1CCC(O)(c2ccccc2)CC1]

(in which the asterisks (*) denote absolute stereochemistry), or is a pharmaceutically acceptable salt thereof.

In some embodiments, the NR2B subunit-selective NMDA antagonist is (±)-cis-4-methylbenzyl 3-fluoro-4-[(pyrimidin-2-ylamino)methyl]piperidine-1-carboxylate (“Compound C”), or a pharmaceutically acceptable salt thereof. In some embodiments, the NR2B subunit-selective NMDA antagonist is (−)-cis-4-methylbenzyl 3-fluoro-4-[(pyrimidin-2-ylamino)-methyl]piperidine-1-carboxylate (“Compound D”), or a pharmaceutically acceptable salt thereof. In some embodiments, the NR2B subunit-selective NMDA antagonist is (+)-cis-4-methylbenzyl-3-fluoro-4-[(pyrimidin-2-ylamino)methyl]piperidine-1-carboxylate, or a pharmaceutically acceptable salt thereof.

In some embodiments, the NR2B subunit-selective NMDA antagonist is (3S,4R)-4-methylbenzyl 3-fluoro-4-[(pyrimidin-2-ylamino)methyl]piperidine-1-carboxylate:

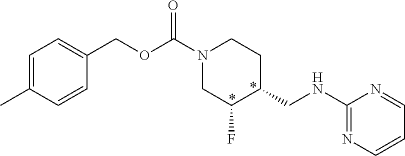 [SMILES: Cc1ccc(COC(=O)N2CC[C@H](CNc3ncccn3)[C@H](F)C2)cc1]

(in which the asterisks (*) denote absolute stereochemistry), or a pharmaceutically acceptable salt thereof. In some embodiments, the NR2B subunit-selective NMDA antagonist is (3R,4S)-4-methyl-benzyl 3-fluoro-4-[(pyrimidin-2-ylamino)methyl]piperidine-1-carboxylate:

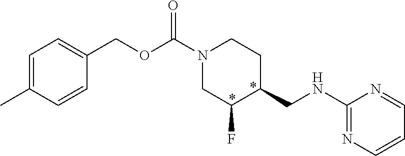 [SMILES: Cc1ccc(COC(=O)N2CC[C@@H](CNc3ncccn3)[C@@H](F)C2)cc1]

(in which the asterisks (*) denote absolute stereochemistry), or a pharmaceutically acceptable salt thereof.

In some embodiments, the NR2B subunit selective NMDA inhibitor is N-(1-(2,2-difluoro-2-(4-tri-fluoromethyl)phenyl-ethyl)piperidin-4-yl)-1H-pyrazolo[3,4-d]pyrimidin-4-amine (“Compound B”):

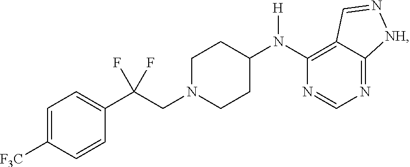 [SMILES: FC(F)(F)c1ccc(C(F)(F)CN2CCC(Nc3ncnc4[nH]ncc34)CC2)cc1]

or a pharmaceutically acceptable salt thereof.

### BRIEF DESCRIPTION OF THE DRAWING

FIGS. 1A and 1B show the novelty phobia behavior of Shank3-KO mice (FIG. 1A) and Sapap3-KO mice (FIG. 1B) as described in Example 2.2.

FIG. 2 shows effects of Compounds A and B on the novelty phobia behavior of Shank3-KO mice as described in Example 2.2.

FIG. 3 shows dose-dependent effects of Compound B on the novelty phobia behavior of Shank3-KO mice as described in Example 2.2.

FIG. 4 shows dose-dependent effects of Compound B on the novelty phobia behavior of Sapap3-KO mice as described in Example 2.2.

FIG. 5 shows dose-dependent effects of Compound C on the novelty phobia behavior of Sapap3-KO mice as described in Example 2.2.

FIG. 6 shows effects of Compound D on the novelty phobia behavior of Sapap3-KO mice as described in Example 2.2.

FIG. 7 shows dose-dependent effects of Compound D on the marble burying behavior of wild-type mice as described in Example 2.2.

FIG. 8 shows dose-dependent effects of Compound D on wild-type mice in the elevated plus maze test as described in Example 2.2.

FIG. 9 shows the asymmetric unit of (−)-4-methylbenzyl 3-fluoro-4-[(pyrimidin-2-ylamino)-methyl]piperidine-1-carboxylate as determined in Example 3.1.

### DETAILED DESCRIPTION OF CERTAIN EMBODIMENTS

In some embodiments, the present invention encompasses the insight that certain NR2B subtype-selective NMDA receptor antagonists are useful in the treatment of autism spectrum disorders (ASDs) and/or anxiety disorders. More particularly, it has been discovered that certain antagonists of NR2B subtype-selective NMDA receptors can correct behavioral abnormalities in animals genetically engineered to model human symptoms associated with ASDs and/or anxiety. Among other thing, the present disclosure establishes that administration of these compounds to individuals suffering from or susceptible to (e.g., diagnosed with, displaying one or more symptoms associated with, genetically related to one or more individuals diagnosed with or displaying one or more symptoms associated with, etc.) one or more ASDs or anxiety disorders has the potential to treat (e.g., reduce frequency and/or severity of, and/or delay onset of, etc) at least one of the behavioral or psychological symptoms associated with such conditions.

### Definitions and Usage

Unless otherwise indicated, the word “includes” (or any variation thereon, e.g., “include”, “including”, etc.) is intended to be open-ended. For example, “A includes 1, 2 and 3” means that A includes but is not limited to 1, 2 and 3.

Unless otherwise indicated, the phrase “such as” is intended to be open-ended. For example, “A can be a halogen, such as chlorine or bromine” means that A can be, but is not limited to, chlorine or bromine.

An “individual” is a mammal including a human. Mammals include humans; farm animals; sport animals; pets; non-human primates; rodents, such as mice and rats; companion animals, such as dogs and cats.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of an Autism Spectrum Disorder or anxiety disorder, an effective amount of the drug may have the effect in reducing and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, “treatment” or “treating” is an approach for obtaining beneficial or desired results including clinical results. For purposes of this invention, beneficial or desired clinical results include one or more of the following: decreasing the severity and/or frequency of one or more symptoms resulting from or otherwise associated with the disease, increasing the quality of life of those suffering from the disease, decreasing the dose of one or more other medications required to treat the disease, delaying the development of the disease, and/or prolonging survival of individuals.

As used herein, “delaying development of a disease” means to defer, hinder, slow, retard, stabilize, and/or postpone development of the disease (such as an Autism Spectrum Disorder or anxiety disorder). This delay can be of varying lengths of time, depending on the history of the disease and/or individual being treated. As is evident to one skilled in the art, a sufficient or significant delay can, in effect, encompass prevention, in that the individual does not develop the disease.

The term “pharmaceutically acceptable” means a material which is not biologically or otherwise undesirable, i.e., the material may be administered to an individual without causing any undesirable biological effects or interacting in a deleterious manner with any of the components of the composition in which it is contained.

It is understood that aspects and variations of the invention described herein include “consisting” and/or “consisting essentially of” aspects and variations.

As used herein, the singular form “a”, “an”, and “the” includes plural references unless indicated otherwise.

### Pharmacology

### NMDA Receptors

The NMDA (N-methyl-D-aspartate) receptor is commonly found on the plasma membranes of neurons located in the central nervous system. This receptor is distinct in two ways: first, it is both ligand-gated and voltage-dependent; second, it requires co-activation by two ligands: glutamate and glycine. The voltage-dependent activation property of the NMDA receptor results from blockage of the ion channel by extracellular Mg²⁺ ions. This brings about the flow of Na⁺and small amounts of Ca²⁺ ions into neurons while K⁺ flows out of the cells resulting in voltage-dependent activation. Calcium flux through NMDA receptors on neural cells is thought be critical in synaptic plasticity, a cellular mechanism for learning and memory (Li & Tsien, N. Engl. J. Med., 361:302 (2009).

Structurally, NMDA receptors form a heterotetramer made up of two NR1 and two NR2 subunits. A related gene family of NR3A and B subunits has also been identified and appears to have an inhibitory effect on receptor activity. Multiple receptor isoforms with distinct brain distributions and functional properties arise by the selective splicing of NR1 transcripts and differential expression of NR2 subunits. See Ishiiet al, J. Biol. Chem., 268:2836-2843 (1993), and Laurie et al., Mol. Brain Res., 51:23-32 (1997). The various resulting combinations produce a variety of NMDA receptors differing in physiological and pharmacological properties such as ion gating properties, magnesium sensitivity, pharmacological profile, as well as in anatomical distribution.

The NR2 NMDA subunit family is in turn divided into four individual subunit types: NR2A, NR2B, NR2C, and NR2D. These subunits contain binding-sites for the neurotransmitter glutamate and each possesses a different intracellular C-terminal domain that can interact with different sets of intracellular signaling messengers (Ryan & Grant, Nat Rev Neurosci 10:701 (2009). Unlike NR1 subunits, NR2 subunits are expressed differentially across various cell types and control the electro-physiological properties of the NMDA receptor. One particular subunit, NR2B, is mainly present in immature neurons and in extrasynaptic locations. Whereas NR2B is predominantly expressed in the early postnatal brain, the number of NR2A subunits grows as the organism ages, and eventually NR2A subunit expression comes to predominate neural expression in NMDA receptors. Referred to as the “NR2B-NR2A developmental switch,” the change in NR2 subunit expression is significant due to the differential kinetic properties each particular NR2 subunit lends to the receptor (Liu et al., J. Neurosci., 24(40):8885-95 (2004)). The NR2B and NR2A subunits each play different roles in intermediating excitotoxic neuronal death with the developmental switch in subunit composition thought to explain developmental changes in NMDA neurotoxicity (Liu et al., J. Neurosci., 27(11):2846-57 (2007); Zhou & Baudry, J. Neurosci., 26(11):2956-63 (2006)). NR2B gene disruption in mice results in perinatal lethality while deletion of the NR2A gene produces viable mice albeit with impaired hippocampal plasticity (Sprengel et al., Cell, 92:279-289 (1998)).

### NR2B Subunit-Selective NMDA Receptor Antagonists

The NMDA receptor is an ionotropic receptor that facilitates the transfer of electrical signals between neurons in the central nervous system. For neural conduction of action potentials, the NMDA receptor is in an open or “activated” state. NMDA receptor activation occurs when glutamate and glycine bind to the NMDA receptor NR2 and NR1 subunits, respectively. NMDA receptor antagonist compounds inhibit or block the opening of the NMDA receptor channel thereby reducing or preventing glycine/glutamate-mediated excitatory postsynaptic potentials.

Described herein are certain NR2B subunit-selective NMDA antagonists. These antagonists selectively bind to the NR2B subunit and inhibit the activity of the NMDA receptor. In some embodiments, an antagonist described herein binds to a the NR2B subunit of a human NMDA receptor. In some embodiments, an antagonist described herein inhibits the activity of a human NMDA receptor. In some embodiments, an antagonist described herein binds to the NR2B subunit of a non-human primate NMDA receptor. In some embodiments, an antagonist described herein inhibits the activity of a non-human primate NMDA receptor. In some embodiments, the non-human primate is a monkey. In some embodiments, an antagonist described herein binds to the NR2B subunit of a rodent NMDA receptor. In some embodiments, an antagonist described herein inhibits the activity of a rodent NMDA receptor. In some embodiments, the rodent is a mouse. In some embodiments, the rodent is a rat. In some embodiments, the precursor of the NR2B subunit comprises the amino acid sequence shown in GenBank accession number NP_000825.

In some embodiments, the NR2B subunit selective NMDA antagonist is traxoprodil (CP-101,606), which is (+)-(1S,2S)-1-(4-hydroxyphenyl)-2-(4-hydroxy-4-phenylpiperidino)-1-propanol:

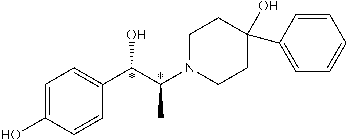 [SMILES: C[C@@H]([C@@H](O)c1ccc(O)cc1)N1CCC(O)(c2ccccc2)CC1]

(in which the asterisks (*) denote absolute stereochemistry), or pharmaceutically acceptable salt thereof.

In some embodiments, the NR2B subunit-selective NMDA antagonist is (±)-cis-4-methylbenzyl 3-fluoro-4-[(pyrimidin-2-ylamino)methyl]piperidine-1-carboxylate, or a pharmaceutically acceptable salt thereof. In some embodiments, the NR2B subunit-selective NMDA antagonist is (−)-cis-4-methyl-benzyl 3-fluoro-4-[(pyrimidin-2-ylamino)methyl]piperidine-1-carboxylate, or a pharmaceutically acceptable salt thereof. In some embodiments, the NR2B subunit-selective NMDA antagonist is (+)-cis-4-methylbenzyl 3-fluoro-4-[(pyrimidin-2-ylamino)methyl]piperidine-1-carboxylate, or a pharmaceutically acceptable salt thereof.

In some embodiments, the NR2B subunit-selective NMDA antagonist is (3S,4R)-4-methylbenzyl 3-fluoro-4-[(pyrimidin-2-ylamino)methyl]piperidine-1-carboxylate:

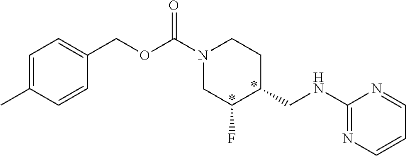 [SMILES: Cc1ccc(COC(=O)N2CC[C@H](CNc3ncccn3)[C@H](F)C2)cc1]

(in which the asterisks (*) denote absolute stereochemistry), or a pharmaceutically acceptable salt thereof. In some embodiments, the NR2B subunit-selective NMDA antagonist is (3R,4S)-4-methyl-benzyl 3-fluoro-4-[(pyrimidin-2-ylamino)methyl]piperidine-1-carboxylate:

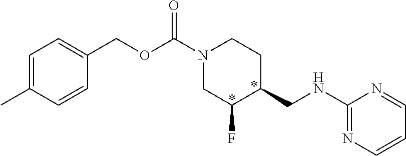 [SMILES: Cc1ccc(COC(=O)N2CC[C@@H](CNc3ncccn3)[C@@H](F)C2)cc1]

(in which the asterisks (*) denote absolute stereochemistry), or a pharmaceutically acceptable salt thereof.

As demonstrated in Example 3.1, in some embodiments the present invention includes the discovery that (−)-4-methylbenzyl 3-fluoro-4-[(pyrimidin-2-ylamino)methyl]piperidine-1-carboxylate has the absolute configuration shown above, i.e., that the (−) enantiomer is (3S,4R)-4-methylbenzyl 3-fluoro-4-[(pyrimidin-2-ylamino)methyl]piperidine-1-carboxylate; and accordingly that the (+) enantiomer is (3R,4S)-4-methylbenzyl 3-fluoro-4-[(pyrimidin-2-ylamino)methyl]piperidine-1-carboxylate.

In some embodiments, the NR2B subunit-selective NMDA antagonist is N-(1-(2,2-difluoro-2-(4-trifluoromethyl)phenyl-ethyl)piperidin-4-yl)-1H-pyrazolo[3,4-d]pyrimidin-4-amine:

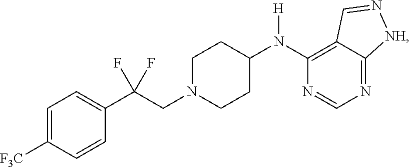 [SMILES: FC(F)(F)c1ccc(C(F)(F)CN2CCC(Nc3ncnc4[nH]ncc34)CC2)cc1]

or a pharmaceutically acceptable salt thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., J. Pharmaceutical Sciences, 1977, 66:1-19 (the entirety of which is incorporated by reference herein) describes pharmaceutically acceptable salts in detail. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Mental Disorders

### Autism Spectrum Disorders

Autism spectrum disorders constitute a range of multifaceted neurodevelopment disorders which are characterized by social impairments, communication difficulties, and restricted, repetitive, and stereotyped patterns of behavior. Autistic disorder (a.k.a. “autism” or “classical ASD”), is the most severe form of ASD, while other conditions along the spectrum include a milder form known as Asperger syndrome, and childhood disintegrative disorder and pervasive developmental disorder not otherwise specified (usually referred to as PDD-NOS). While ASD can differ significantly in character and severity depending on the individual, it is known to occur in all ethnic and socioeconomic groups and in all age groups. According to one estimate, 1 out of 88 children age 8 have an ASD (Centers for Disease Control and Prevention: Morbidity and Mortality Weekly Report, Mar. 30, 2012) with males are more likely to have an ASD in comparison to females.

Individuals with ASDs commonly have difficulties in three key areas of development—social interaction, language and behavior. However, due to the fact that ASD symptoms vary widely, two individuals with identical diagnoses may behave quite differently as outward manifestations of the condition. In many cases, however, the most severe cases of ASD are marked by the complete inability to communicate or interact with other people. Some individuals show signs of ASD in early infancy while others may develop normally for the first few months or years of life but then suddenly become withdrawn, aggressive or lose language skills that have been already acquired. Although each individual with autism is likely to exhibit a pattern of behavior unique to that individual, there are some common symptoms associated with ASDs, falling into categories of social skills, behavior, and language.

Social impairment and the failure to develop social relationships are hallmarks of ASDs. These can include an individual failing to respond to his or her name, inability to maintain eye contact with another person, inability to respond when another person is speaking to the individual, resistance to intimate parent-child behavior such as cuddling, hugging, or holding, inability to appreciate the feelings of other (i.e. lack of empathy), and a preference to be alone or to play alone.

Additionally, individuals with an ASD commonly exhibit one or more characteristic behaviors. These can include the performance of repetitive movements (such as rocking, spinning or hand-flapping), behavioral rigidity (such as the development and repetitive performance of specific routines or rituals as well as agitation upon the slightest change in these routines or rituals), constant movement, excessive lining up of toys or objects, lack of smiling, restricted patterns of interest that are abnormal in intensity or focus, unusual sensitivity to light, sound and touch, and obliviousness to pain. Additionally, individuals with ASDs often exhibit repetitive and disruptive behaviors which may result from exposure to novel or unexpected events and experiences, particularly ones which disrupt a set routine. Indeed, it is believed that novelty itself is disagreeable to individuals with ASDs and that novelty avoidance might cause attention impairments and, as a consequence, result in autistic characteristics (Dawson et al., 1998, J. Autism and Dev. Disorders, 28:479-485).

Defects in language acquisition and communication are also characteristic symptoms of ASDs. These can include failure to acquire language (including no babbling or pointing by age 1, no use of single words by 16 months or use of two-word phrases by age 2), loss of previously acquired ability to use words or sentences, failure to make eye contact when making requests, speaking with an abnormal tone of voice or rhythm (such as a singsong voice or “robot-sounding” speech), inability to start a conversation or keep one going, repetition of words or phrases verbatim, but without the understanding of how to use them. Other symptoms of ASDs include inability to make friends with peers, absence or impairment of imaginative and social play, stereotyped or unusual use of language, and a preoccupation with particular objects or subjects.

Presently, there is no medical test that capable of diagnosing autism spectrum disorders. Accordingly, diagnosis of ASDs is based on behavior, not cause or mechanism (London, Brain Pathol., 2007; 17(4):408-11). The method of diagnosis varies, with some health care providers using available questionnaires or other screening instruments to gather information about an individual's development and behavior. For example, one commonly used screening device is known as “The Modified Checklist for Autism in Toddlers (M-CHAT™)” which is a scientifically validated tool for screening children between 16 and 30 months of age to assess their risk for ASD. Other screening devices rely solely on parental observations, while others rely on a combination of parent and doctor observations. If these screening instruments indicate the possibility of an ASD, a more comprehensive evaluation is typically undergone. A comprehensive evaluation usually employs several health care providers that can include psychologists, neurologists, psychiatrists, speech therapists, and other professionals who diagnose children with ASDs who conduct neurological assessments as well as cognitive and language testing.

Individuals exhibiting some symptoms of an ASD but lacking the number required to be diagnosed with classical autism are often diagnosed with pervasive developmental disorder not otherwise specified (PDD-NOS). Individuals with autistic behaviors and autistic impairments in social skills but who nevertheless have well-developed language skills are often diagnosed with Asperger syndrome. Much rarer are children who may be diagnosed with childhood disintegrative disorder, in which they develop normally and then suddenly deteriorate between the ages of 3 to 10 years and show marked autistic behaviors.

### Anxiety Disorders

Anxiety disorders represent an array of psychiatric conditions characterized by excessive and debilitating worry or anxiousness over specific or non-specific life events or situations. These feelings often have notable physical manifestations, including particular panic or stress reactions. Individual anxiety disorders are sometimes defined by the specific source of the anxiety (e.g., post-traumatic stress disorder, social phobia, or specific phobia). Other classifications are based on the type of reaction (e.g., obsessive-compulsive disorder, agoraphobia, panic disorder, generalized anxiety disorder). It is common for patients to experience a variety of symptoms and therefore be diagnosed with multiple types of anxiety disorders. Conventional treatment includes both psychotherapy and a subset of neuroactive drugs, including modulators of the serotonin or GABA receptor systems. Up to half of the patients do not respond adequately to treatment or cannot tolerate the side effects of drug therapy.

Agoraphobia and panic disorder. These types of anxiety disorders share the symptom of panic attack. Panic attack is characterized by discrete instances of intense fear or discomfort in the absence of any real danger (Diagnostic and Statistical Manual of Mental Disorders IV, American Psychiatric Association, 2000, “DSM-IV-TR®”). The attack is accompanied by at least 4 somatic or cognitive symptoms out a list of 13 (palpitations, pounding heart, or accelerated heart rate; sweating; trembling or shaking; sensations of shortness of breath or smothering; feeling of choking; chest pain or discomfort; nausea or abdominal distress; feeling dizzy, unsteady, lightheaded or faint; feelings of unreality (derealization) or being detached from oneself (depersonalization); fear of losing control or going crazy; fear of dying numbness or tingling sensations (paresthesias); chills or hot flushes). The attacks have very sudden onset peak quickly.

Agoraphobia is defined as worry or excessive avoidance of events, places or situations that might trigger a panic attack. This fear leads to pervasive avoidance behaviors of situations thought to trigger the panic attacks. Situations avoided can include being alone, being outside of home, traveling in cars or airplanes, or being in elevators or bridges. The anxiety caused by fear of these situations can impair normal activities and cause substantial debilitation.

Panic disorder is defined as recurrent unexpected panic attacks and at least one of the attacks have been followed by 1 month (or more) of one (or more) of the following: the attacks are not due to the direct physiological effects of a substance (such as drug of abuse or a medication), or a general medical condition; the attacks are not better accounted for by another mental disorder, such as social phobia (such as occurring on exposure to feared social situations), specific phobia, obsessive-compulsive disorder, post-traumatic stress disorder or separation anxiety disorder. Panic disorders can be present with or without agoraphobia and agoraphobia can occur without previous history of panic disorder. The prevalence of panic disorder with or without agoraphobia is thought to be approximately 1-2% with some evidence of a genetic or familial component in up to 25% of the cases.

Social phobia. The primary characteristic of social phobia is marked fear of embarrassment in typical social or performance situations, including meeting new people, public speaking, group activities, or other common public activities. These situations routinely generate symptoms similar to those of panic attack with a range of severities, and individuals often avoid these situations or face them with substantial fear and worry. The fear and avoidance of these situations can interfere significantly with normal life activities, job or school performance, and personal relationships. The lifetime prevalence of social phobia meeting DSM-IV-TR diagnostic criteria is thought to be around 2%. There is some evidence of genetic or familial association, but this is consider relatively modest.

Post-traumatic stress disorder. PTSD is characterized by chronic and repeated anxiety or fear generated by a previous, well-defined traumatic experience. The traumatic experience is severe in nature and typically entails an event that threaten death or serious injury either in the patient or a close relative or associate. The event elicits a response of fear, horror, or helplessness and generates an ongoing set of anxiety-like symptoms that are associated with disability and disruption to normal life experiences. Symptoms include persistent re-experiencing of the event, avoidance of stimuli associated with the traumatic event, numbing of general responsiveness, and increased arousal. Symptoms must persist for at least one month to meet diagnostic criteria. In most but not all cases, a major feature involves the patient going into a dissociative state lasting from seconds to days in which they relive the traumatic event or believe those events are happening again. These “flashbacks” are often triggered by stimuli that may resemble or remind the person of the original event. Thus, possible exposure to such stimuli are persistently and actively avoided causing significant degradation of quality-of-life. The lifetime prevalence of PTSD in the US is estimated to be 8% of the adult population. The propensity for suffering PTSD may have a familial or genetic component.

Generalized anxiety disorder. Generalized anxiety disorder (GAD) is a type of anxiety disorder characterized by excessive anxiety and worry about multiple events or activities that occurs more days than not for at least 6 months. GAD has a lifetime prevalence of approximately 5% with an annual economic burden of $50 billion in the USA alone (H. U. Wittchen, Depress Anxiety 2002; 14:162-67). Approximately 25% of patients in anxiety disorder clinics have a presenting or comorbid diagnosis of GAD. Twin studies have shown a clear genetic association for the presence of GAD symptoms (J. M. Hettema et al., Am. J. Psychiatry 2001; 158:1568-78; S. I. Chantarujikapong et al., Psychiatry Res. 2001; 103:133-45). GAD patients most often report they have felt anxious for most of their lives, that it fluctuates in severity but worsens during times of stress.

GAD patients show worry and anxiety that they are not able to control and are accompanied by at least three of the following symptoms: restlessness, being easily fatigued, difficulty concentrating, irritability, muscle tension, and disturbed sleep. The focus of the worry is generally everyday, routine life circumstances, and the intensity and duration is out of proportion to the impact or likelihood of the event. Individuals with GAD report subjective distress due to excessive worry that they cannot control, which impairs normal life activities such as social interactions and job.

Obsessive-compulsive disorder. Obsessive compulsive disorder (OCD) is a type of anxiety disorder defined by pervasive and intrusive thoughts that produce discomfort, nervousness, fear, or anxiety, by repetitive and compulsive behaviors aimed at reducing the associated anxiety, or by a combination of such obsessions and compulsions. OCD is the fourth most common mental disorder and is believed to have a lifetime prevalence of about 2.5%, though other estimates of lifetime prevalence are generally in the range of 1.7-4%. (M. Karno et al., Arch Gen Psychiatry 1988; 45(12):1094-99). According to the Diagnostic and Statistical Manual of Mental Disorders IV (American Psychiatric Association, 1994, “DSM-IV®”), obsessions and compulsions in OCD are discrete phenomena that may or may not co-occur. In particular, obsessions are defined as repetitive or intrusive thoughts, impulses, or images that cause marked anxiety or distress. Compulsions, on the other hand, are considered to be repetitive and ritualistic behavior or mental acts that the individual feels compelled to accomplish, whose purpose is to reduce distress, but that are not realistically connected with that distress.

Individuals with OCD typically exhibit symptoms of both intrusive thoughts and compulsive behaviors. However, in the case of so-called “Purely Obsessional Obsessive-Compulsive Disorder” (a.k.a. OCD without overt compulsions or Primarily Obsessional OCD), the individual does not exhibit the compulsive behaviors normally observed in typical OCD. Nevertheless, even in this variant OCD disorder, ritualizing and neutralizing behaviors do take place, though they are almost entirely in the form of excessive repetitious thoughts (Toates & Coschug-Toates, Obsessive compulsive disorder, (2nd Ed.: 2000), pp. 111-128). The intrusive thoughts characteristic of individuals having or suspected of having OCD can take many varied forms, depending on the individual. These thoughts are usually involuntary in nature and consist of mental images and unpleasant ideas that are upsetting or distressing and which can be difficult to manage or eliminate. When occurring in OCD, individuals are less likely to be able to ignore these thoughts and end up paying excessive attention to them, resulting in the thoughts becoming ever more distressing and frequently occurring. In some individuals with OCD, these thoughts can be paralyzing, severe, and constantly present, and often center around inappropriately aggressive thoughts (e.g., violent obsessions about hurting themselves or other people), inappropriately sexual thoughts (e.g., intrusive thoughts or images of sexual activity and/or rape with strangers, friends, or family members as well as persistent thoughts relating to sexual orientation), or blasphemous thoughts pertaining to religious activities or subjects.

To be diagnosed with anxiety disorders, typically a person must meet the particular diagnostic criteria described above and further outlined in DSM-IV®. In most cases the symptoms must be present for weeks to months and cause significant disruption of the individuals normal functioning or wellness. Behaviors associated with the anxious state generally must be out of proportion to the reported source of the worry and fear or the initial triggering event causing the problem. While there is no single medical test capable of diagnosing anxiety disorders and identifying the specific subtype, diagnosis may be performed by a psychologist, psychiatrist, clinical social worker, or other licensed mental health professional. In some instances, rating scales are available (such as the Yale-Brown Obsessive Compulsive Scale (“Y-BOCS”) for OCD or the Hamilton Anxiety Scale (“HAM-A”) for GAD to assist the mental health professional in diagnosing the individual.

### Use, Formulation and Administration of NR2B Subunit-selective NMDA Receptor Antagonists

### Uses of NR2B Subunit-selective NMDA Receptor Antagonists and Pharmaceutically Acceptable Compositions

The present invention provides methods for treating autism spectrum disorders (ASDs) and/or anxiety disorders by administering an effective amount of a described NR2B subunit-selective NMDA receptor antagonist (“antagonist”). The antagonist can be administered in any amount and using any route of administration effective for treating the disorder. In some embodiments, the antagonist is administered as a pharmaceutical composition, as described herein.

### Anxiety Disorders

In some embodiments, the present invention provides a method for treating an anxiety disorder in an individual in need thereof comprising administering an effective amount of a described NR2B subunit-selective NMDA receptor antagonist. The individual can be one diagnosed with an anxiety disorder or one suspected by a treating physician of having an anxiety disorder. In some embodiments, the individual is one diagnosed with an anxiety disorder. In some embodiments, the individual is a human.

In some embodiments, the anxiety disorder is obsessive-compulsive disorder, generalized anxiety disorder, agoraphobia (with or without panic disorder), panic disorder, post-traumatic stress disorder or social anxiety disorder, and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)-methyl]piperidine-1-carboxylate, or N-(1-(2,2-difluoro-2-(4-trifluoromethyl)phenyl-ethyl)piperidin-4-yl)-1H-pyrazolo[3,4-d]pyrimidin-4-amine, or a pharmaceutically acceptable salt thereof.

In some embodiments, the anxiety disorder is obsessive-compulsive disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-tri-fluoromethyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-yl-amino)methyl]piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]-piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methyl-benzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoro-methyl)phenyl-ethyl)piperidin-4-yl)-1H-pyrazolo[3,4-d]pyrimidin-4-amine or a pharmaceutically acceptable salt thereof.

In some embodiments, the anxiety disorder is generalized anxiety disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoro-methyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-ylamino)methyl]-piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methyl-benzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoromethyl)phenyl-ethyppiperidin-4-yl)-1H-pyrazolo[3,4-d]pyrimidin-4-amine or a pharmaceutically acceptable salt thereof.

In some embodiments, the anxiety disorder is agoraphobia with panic disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoromethypphenyl-ethyppiperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-yl-amino)methyl]piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]-piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)methyl]piperidine-1-carboxylate or a pharmaceutically acceptable salt thereof. In some embodiments, the antagonist is N-(1-(2,2-di-fluoro-2-(4-trifluoromethyl)phenyl-ethyl)piperidin-4-yl)-1H-pyrazolo[3,4-d]pyrimidin-4-amine or a pharmaceutically acceptable salt thereof.

In some embodiments, the anxiety disorder is agoraphobia without panic disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-di-fluoro-2-(4-trifluoromethyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-yl-amino)methyl]piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]-piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methyl-benzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoro-methyl)phenyl-ethyl)piperidin-4-yl)-1H-pyrazolo[3,4-d]pyrimidin-4-amine or a pharmaceutically acceptable salt thereof.

In some embodiments, the anxiety disorder is panic disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoro-methyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-ylamino)methyl]-piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methyl-benzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoromethyl)phenyl-ethyl)piperidin-4-yl)-1H-pyrazolo[3,4-d]pyrimidin-4-amine or a pharmaceutically acceptable salt thereof.

In some embodiments, the anxiety disorder is post-traumatic stress disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-tri-fluoromethyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-yl-amino)methyl]piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]-piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methyl-benzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoro-methyl)phenyl-ethyl)piperidin-4-yl)-1H-pyrazolo[3,4-d]pyrimidin-4-amine or a pharmaceutically acceptable salt thereof.

In some embodiments, the anxiety disorder is social anxiety disorder and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoro-methyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-ylamino)methyl]-piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methyl-benzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoromethyl)phenyl-ethyl)piperidin-4-yl)-1H-pyrazolo[3,4-d]pyrimidin-4-amine or a pharmaceutically acceptable salt thereof.

In some embodiments, a described NR2B subunit-selective NMDA receptor antagonist is administered with one or more therapeutic agents useful for treating an anxiety disorder. Such therapeutic agents include agents for treating any of the symptoms of anxiety disorders, such as those described herein, e.g., excessive worry or anxiety. In some embodiments, a therapeutic agent useful for treating an anxiety disorder is a selective serotonin reuptake inhibitor, a tri-cyclic antidepressant, a benzodiazepine, an atypical antipsychotic or a serotonin-norepinephrine reuptake inhibitor. Examples of such therapeutic agents useful in the provided methods include selective serotonin reuptake inhibitors (such as Citalopram, Dapoxetine, Escitalopram, Fluoxetine, Fluvoxamine, Indalpine, Paroxetine, Sertraline, Vilazodone and Zimelidine), tricyclic antidepressants (such as Amitriptyline, Amitriptylinoxide, Butriptyline, Clomipramine, Demexiptiline, Desipramine, Dibenzepin, Dimetacrine, Dosulepin/Dothiepin, Doxepin, Imipramine, Imipraminoxide, Lofepramine, Melitracen, Metapramine, Nitroxazepine, Nortriptyline, Noxiptiline, Pipofezine, Propizepine, Protriptyline, Quinupramine, Amineptine, Iprindole, Opipramol, Tianeptine and Trimipramine), benzodiazepines (such as Alprazolam, Bretazenil, Bromazepam, Brotizolam, Chlordiazepoxide, Cinolazepam, Clonazepam, Clorazepate, Clotiazepam, Cloxazolam, Delorazepam, Diazepam, Estazolam, Etizolam, Flunitrazepam, Flurazepam, Flutoprazepam, Halazepam, Ketazolam, Loprazolam, Lorazepam, Lormetazepam, Medazepam, Midazolam, Nimetazepam, Nitrazepam, Nordazepam, Oxazepam, Phenazepam, Pinazepam, Prazepam, Premazepam, Quazepam, Temazepam, Tetrazepam, Triazolam, Clobazam, DMCM (methyl-6,7-dimethoxy-4-ethyl-beta-carboline-3-carboxylate), Zaleplon, Zolpidem and Zopiclone), atypical antipsychotics (such as Amisulpride, Aripiprazole, Asenapine, Blonanserin, Clotiapine, Clozapine, Iloperidone, Llurasidone, Mosapramine, Olanzapine, Paliperidone, Perospirone, Quetiapine, Remoxipride, Risperidone, Sertindole, Sulpiride, Ziprasidone, Zotepine, Bifeprunox, Pimavanserin and Vabicaserin), inositol, μ-Opioids (such as hydrocodone and tramadol), serotonin-norepinephrine reuptake inhibitors (such as venlafaxine and duloxetine), anticholinergics (such as Benztropine, Ispratropium, Oxitropium, Tiotropium, Glycopyrrolate, Oxybutinin, Tolterodine, Diphenhydramine and Dimenhydrinate) and nicotine.

In some embodiments, a described NR2B subunit-selective NMDA receptor antagonist is administered in conjunction with a behavioral-based, psychosocial-based, or psychological-based therapy useful for the treatment of anxiety disorders. Examples of such behavioral-based, psychosocial-based, or psychological-based therapy for treatment of an anxiety disorder useful in the provided methods include behavioral therapy (e.g., exposure and response prevention), cognitive behavioral therapy, and psychodynamic psychotherapy.

In some embodiments, a described NR2B subunit-selective NMDA receptor antagonist is administered in conjunction with electroconvulsive therapy or psychosurgery (e.g., deep brain stimulation or vagus nerve stimulation). Such therapies are particularly useful in severe and/or refractory cases of anxiety disorders.

### Autism Spectrum Disorders

In some embodiments, the present invention provides a method for treating an autism spectrum disorder (ASD) in an individual in need thereof comprising administering an effective amount of a described NR2B subunit-selective NMDA receptor antagonist. The individual can be one diagnosed with an ASD or one suspected by a treating physician of having an ASD. In some embodiments, the individual is one diagnosed with an ASD. In some embodiments, the individual is a human.

In some embodiments, the ASD is autism, Asperger's syndrome or pervasive developmental disorder not otherwise specified (PDD-NOS), and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)-methyl]piperidine-1-carboxylate, or N-(1-(2,2-difluoro-2-(4-trifluoromethypphenyl-ethyl)piperidin-4-yl)-1H-pyrazolo[3,4-d]pyrimidin-4-amine, or a pharmaceutically acceptable salt thereof.

In some embodiments, the ASD is autism and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)-methyl]piperidine-1-carboxylate, or N-(1-(2,2-difluoro-2-(4-trifluoromethypphenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-ylamino)methyl]piperidine-1-carboxylate or a pharmaceutically acceptable salt thereof. In some embodiments, the antagonist is (−)-cis-4-methyl-benzyl 3-fluoro-4-[(pyrimidin-2-ylamino)methyl]piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)-methyl]piperidine-1-carboxylate or a pharmaceutically acceptable salt thereof. In some embodiments, the antagonist is N-(1-(2,2-difluoro-2-(4-trifluoromethyl)phenyl-ethyl)piperidin-4-yl)-1H-pyrazolo[3,4-d]pyrimidin-4-amine or a pharmaceutically acceptable salt thereof.

In some embodiments, the ASD is Asperger's syndrome and the antagonist is (+)-(1S,2S)-1-(4-hydroxyphenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoro-methyl)phenyl-ethyl)piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenylpiperidino)-1-propanol or a pharmaceutically acceptable salt thereof. In some embodiments, the antagonist is cis-4-methylbenzyl 3-fluoro-4-[(pyrimidin-2-ylamino)methyl]-piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]piperidine-1-carboxylate or a pharmaceutically acceptable salt thereof. In some embodiments, the antagonist is (+)-cis-4-methylbenzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoromethypphenyl-ethyppiperidin-4-yl)-1H-pyrazolo[3,4-d]pyrimidin-4-amine or a pharmaceutically acceptable salt thereof.

In some embodiments, the ASD is PDD-NOS and the antagonist is (+)-(1S,2S)-1-(4-hydroxy-phenyl)-2-(4-hydroxy-4-phenylpiperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoromethyl)phenyl-ethyl)-piperidin-4-yl)-1H-pyrazolo[3,4-d]pyrimidin-4-amine, or a pharmaceutically acceptable salt thereof. In some embodiments, the antagonist is (+)-(1S,2S)-1-(4-hydroxyphenyl)-2-(4-hydroxy-4-phenyl-piperidino)-1-propanol or a pharmaceutically acceptable salt thereof. In some embodiments, the antagonist is cis-4-methylbenzyl 3-fluoro-4-[(pyrimidin-2-ylamino)methyl]piperidine-1-carboxylate or a pharmaceutically acceptable salt thereof. In some embodiments, the antagonist is (−)-cis-4-methylbenzyl 3-fluoro-4-[(pyrimidin-2-ylamino)methyl]piperidine-1-carboxylate or a pharmaceutically acceptable salt thereof. In some embodiments, the antagonist is (+)-cis-4-methyl-benzyl 3-fluoro-4-[(pyrimidin-2-ylamino)methyl]piperidine-1-carboxylate or a pharmaceutically acceptable salt thereof. In some embodiments, the antagonist is (3S,4R)-4-methylbenzyl 3-fluoro-4-[(pyrimidin-2-ylamino)methyl]piperidine-1-carboxylate or a pharmaceutically acceptable salt thereof. In some embodiments, the antagonist is (3R,4S)-4-methylbenzyl 3-fluoro-4-[(pyrimidin-2-ylamino)methyl]piperidine-1-carboxylate or a pharmaceutically acceptable salt thereof. In some embodiments, the antagonist is N-(1-(2,2-difluoro-2-(4-trifluoromethyl)phenyl-ethyl)piperidin-4-yl)-1H-pyrazolo[3,4-d]pyrimidin-4-amine or a pharmaceutically acceptable salt thereof.

In some embodiments, a described NR2B subunit-selective NMDA receptor antagonist is administered with one or more therapeutic agents useful for treating an ASD. Such therapeutic agents include agents for treating any of the symptoms of ASDs, such as those described herein, e.g., novelty phobia, behavioral rigidity as well as defects in social skills, behavior, and language use/acquisition. In some embodiments, a therapeutic agent useful for treating an ASD is a selective serotonin reuptake inhibitor, a typical antipsychotic, an atypical antipsychotic, a serotonin-norepinephrine reuptake inhibitor, a stimulant, a dopamine receptor agonist, secretin or oxytocin. Examples of such therapeutic agents useful in the provided methods include selective serotonin reuptake inhibitors (such as Citalopram, Dapoxetine, Escitalopram, Fluoxetine, Fluvoxamine, Indalpine, Paroxetine, Sertraline, Vilazodone and Zimelidine), typical antipsychotics (such as Chlorpromazine, Thioridazine, Mesoridazine, Levomepromazine, Loxapine, Molindone, Perphenazine, Thiothixene, Trifluoperazine, Haloperidol, Fluphenazine, Droperidol, Zuclopenthixol, Flupentixol and Prochlorperazine), atypical antipsychotics (such as Amisulpride, Aripiprazole, Asenapine, Blonanserin, Clotiapine, Clozapine, Iloperidone, Llurasidone, Mosapramine, Olanzapine, Paliperidone, Perospirone, Quetiapine, Remoxipride, Risperidone, Sertindole, Sulpiride, Ziprasidone, Zotepine, Bifeprunox, Pimavanserin and Vabicaserin), serotonin-norepinephrine reuptake inhibitors (such as venlafaxine and duloxetine), anticonvulsants (such as Tegretol, Lamictal, Topamax or Depakote), stimulants (such as Adderall, Concerta, Dexedrine, Focalin, Metadate, Methylin, Ritalin and Vyvanse), dopamine receptor agonists (such as Aplindore, Apomorphine, Bromocriptine, Cabergoline, Ciladopa, Dihydroergocryptine, Lisuride, Pardoprunox, Pergolide, Piribedil, Pramipexole, Ropinirole and Rotigotine), secretin and oxytocin.

In some embodiments, a described NR2B subunit-selective NMDA receptor antagonist is administered in conjunction with a behavioral-based, psychosocial-based, or psychological-based therapy for treatment of an ASD. Intensive, sustained special education programs and behavioral therapy are commonly utilized to help individuals diagnosed with or suspected of having an ASD acquire self-care, social and job skills, improved functioning, and decreased symptom severity and maladaptive behavior. Examples of such behavioral-based, psychosocial-based, or psychological-based therapy for treatment of an ASD useful in the provided methods include behavior analysis, early intensive behavior intervention (EIBI; a.k.a. the LOVAAS method), pivotal response therapy, aversion therapy, the social communication, emotional regulation, transactional support (SCERTS) model, relationship development intervention, sensory integration, massage therapy, animal-assisted therapy, neurofeedback, patterning, packing, developmental model-based therapy, structured teaching, speech and language therapy, social skills therapy, and occupational therapy.

### Formulation and Administration of NR2B Subunit-selective NMDA Receptor Antagonists and Pharmaceutically Acceptable Compositions

In some embodiments, the present invention provides administration of a composition comprising a described NR2B subunit-selective NMDA receptor antagonist (“antagonist”) and a pharmaceutically acceptable carrier, adjuvant or vehicle (together, an “antagonist composition”) to an individual in need of such a composition. In some embodiments, the individual is a human. The administrations of an antagonist described herein are contemplated to include administration of an antagonist composition.

The term “pharmaceutically acceptable carrier, adjuvant, or vehicle” refers to a non-toxic carrier, adjuvant, or vehicle that does not destroy the pharmacological activity of the antagonist with which it is formulated. Pharmaceutically acceptable carriers, adjuvants or vehicles that can be used in the antagonist compositions include ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Antagonist compositions can be administered orally, parenterally, by inhalation spray, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the antagonist compositions are administered orally. In some embodiments, the antagonist compositions are administered parenterally. In some embodiments, the antagonist compositions are administered intracranially (e.g., via intracereberal or intracerebroventricular injection) or intravenously. Sterile injectable forms of the compositions of this invention can be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Hank's solution, Ringer's solution and isotonic sodium chloride solution. In addition, sterile, fixed oils are conventionally employed as a solvent or suspending medium.

For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents that are commonly used in the formulation of pharmaceutically acceptable dosage forms, including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Antagonist compositions can be orally administered in any orally acceptable dosage form including capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

Antagonist compositions can be administered in the form of suppositories for rectal administration. These can be prepared by mixing the agent with a suitable non-irritating excipient that is solid at room temperature but liquid at rectal temperature and will therefore melt in the rectum to release the drug. Such materials include cocoa butter, beeswax and polyethylene glycols.

Antagonist compositions can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

Antagonist compositions can be administered by oral administration. Such formulations can be administered with or without food. In some embodiments, antagonist compositions are administered without food. In some embodiments, antagonist compositions are administered with food.

In some embodiments, the amount of antagonist administered to the individual in one day is about 0.01 to about 20 mg/kg body weight, e.g., 0.05 to about 15 mg/kg, about 0.05 mg/kg, about 0.1 mg/kg, about 0.15 mg/kg, about 0.25 mg/kg, about 0.5 mg/kg, about 0.75 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg or about 15 mg/kg. In some embodiments, the antagonist is administered to the individual daily, every other day, once every three days, once every four days, or once a week. In some embodiments, the subunit selective NMDA receptor antagonist is administered to the individual every day for at least 1, 2, 3, 4, 5, 6, 7, 8, 9, 10, 11, 12, 13, 14, 15, 20, 25, 30, 60, 90, 120, 150, 180, 210, 240, 270, 300, 330 or 360 days. In other embodiments, the antagonist is administered daily, every other day, once every three days, once every four days, or once a week, for as long as administration of the antagonist is associated with reduction or inhibition of one or more symptoms associated with the disorder for which it is administered.

The amount of antagonist that can be combined with the carrier materials to produce a composition in a single dosage form will vary depending upon a variety of factors, including the host treated and the particular mode of administration. In some embodiments, antagonist compositions are formulated so that a dosage of about 0.01 to about 20 mg/kg body weight/day, e.g., about 0.05 to about 15 mg/kg body weight/day, of the antagonist can be administered to a patient receiving these compositions.

It should also be understood that a specific dosage and treatment regimen for any particular patient will depend upon a variety of factors, including the activity of the specific antagonist employed, the age, body weight, general health, sex, diet, time of administration, rate of excretion, drug combination, and the judgment of the treating physician and the severity of the particular disorder being treated. The amount of a compound of antagonist in the composition will also depend upon the particular antagonist in the composition.

It should be appreciated that there may be considerable overlap in function of excipients used in the antagonist compositions described herein. Thus, any categorization of the above-listed excipients should be taken as merely exemplary of the types of excipients that are contemplated for inclusion in the antagonist compositions described herein. Further examples of pharmaceutical excipients suitable for use in the antagonist compositions can be found in the U.S. Pharmacopeia National Formulary, 1990, pp. 1857-1859, as well as in Rowe, et al., Handbook of Pharmaceutical Excipients, 5th ed., 2006, and “Remington: The Science and Practice of Pharmacy,” 21st ed., 2006, editor David B. Troy, and in the Physician's Desk Reference, 52nd ed., Medical Economics, Montvale, N.J., 1998, the applicable contents of which are hereby incorporated by reference herein.

Human Genetics of ASD, Anxiety Disorders such as OCD, and Corresponding Transgenic Animal Models

Psychiatric drug discovery has been markedly enhanced by the development of animal models of ASD and anxiety (e.g., OCD) based on gene knock-outs or related genetic manipulations (Fineberg et al., Br. J. Pharmacol. 164:1044, 2011; Crawley Dialog. Clin. Neurosci. 14:293, 2012; Griebel and Holmes, Nature Rev. Drug Disc. 12:667, 2013). Such models provide an important link between the factors thought to cause human disease and in vivo preclinical testing that leads to new drug therapies. The present invention includes the development and characterization of two mouse models that are specifically designed to investigate the underlying pathologies associated with ASD in the case of the Shank3 knock-out mouse and anxiety (e.g., OCD) in the case of the Sapap3 knock-out mouse. The demonstrated ability of the described compounds to improve the behavioral deficits seen in these modified mice demonstrates that these compounds are capable of treating human patients suffering from ASD and anxiety disorders.

Recent human genetic and genomic studies have identified a number of candidate genes for ASDs, many of which encode glutamatergic synaptic proteins including Shank3, Neuroligin-3, Neuroligin-4 and Neurexin-1. Among those candidate genes, Shank3 showed the strongest genetic linkage with human ASD. Disruption of Shank3 is believed to be the cause of the core neurodevelopmental and neurobehavioral deficits in Phelan-McDermid syndrome (PMS), a disorder in which individuals commonly display several autistic behaviors and symptoms (Wilson et al., 2003, J Med Genet 40, 575-584). Recent genetic screens have identified mutations in the Shank3 gene (including microdeletions, nonsense mutations and recurrent breakpoints) in ASD patients not diagnosed with PMS (Durand et al., 2007, Nat Genet 39, 25-27; Moessner et al., 2007, Am J Hum Genet 81, 1289-1297; Gauthier et al., 2009, Am J Med Genet B Neuropsychiatr Genet 150B, 421-424). Consistent with human genetic studies, deletions of these synaptic genes in mice lead to autistic-like behaviors including repetitive behaviors, excessive self-grooming, social interaction deficits, phobic behaviors and anxiety. Notably, Shank3B knockout mice exhibit increased anxiety, reduced rearing and phobia of novel objects. Together these data suggest that Shank3B knockout mice can serve as a model of ASD with both construct and face validity (Peca et al., 2011, Nature, 472(7344):437-42).

Obsessive compulsive disorder (OCD) is an anxiety spectrum disorder. Like ASD, OCD has a strong genetic component. Recent human genetic studies also linked SAPAP3 to OCD and OCD-spectrum disorders (Züchner et al., 2009, Mol. Psychiatry, 14:6-9; Bienvenu et al., 2009, Am J Med Genet B Neuropsychiatr Genet. 150B(5):710-20; Boardman et al., 2010, Compr Psychiatry. 52(2):181-7; Ryu et al., 2011, Am J Med Genet B Neuropsychiatr Genet. 156B(8):949-59; Crane et al., 2011, Am J Med Genet B Neuropsychiatr Genet. 156B(1):108-14). Mice with genetic deletion of the SAPAP3 gene exhibit increased anxiety and compulsive grooming behavior leading to facial hair loss and skin lesions. Neurophysiological, biochemical and pharmacological studies of SAPAP3 mutant mice demonstrated defects in the striatum, a key brain region that is dysfunctional in OCD patients. Together, these data support SAPAP3 knockout mice as a disease model of OCD (Welch et al., 2007, Nature, 448(7156):894-900).

Shank3 and SAPAP3 are protein partners, and together with PSD95 they form a local scaffold, orchestrating the assembly of the macromolecular postsynaptic signaling complex at glutamatergic synapses. This complex plays important roles in targeting, anchoring, and dynamically regulating synaptic localization of glutamatergic receptors, including NMDA receptors and AMPA receptors, as well as many other signaling molecules. Genetic deletion of the Shank3 and Sapap3 genes result in reduced strength of synaptic connections and decreased efficiency of glutamatergic neurotransmission, which mirror the cellular mechanisms observed in individuals with ASD and OCD. Glutamatergic synapses contain two types of ionotropic glutamate receptors: AMPA receptors and NMDA receptors. AMPA receptors mediate fast synaptic transmission, whereas NMDA receptors play a critical role in regulating synaptic function and plasticity. Most native NMDA receptors (NMDARs) are heterotetramers consisting of two obligatory NR1 subunits and two of the four (NR2A-NR2D) NR2 subunits. Different NR2 subunits have different distribution pattern in the brain and confer distinct properties to the receptors. In much of the adult brain, typical mature synapses contain mainly NMDARs with NR2A, while NR2B-containing NMDARs are common in the extrasynaptic membrane area. The extrasynaptic NR2B NMDAR plays an important role in regulating synaptic strength and the trafficking of AMPA receptor and NMDA receptor at the synaptic site.

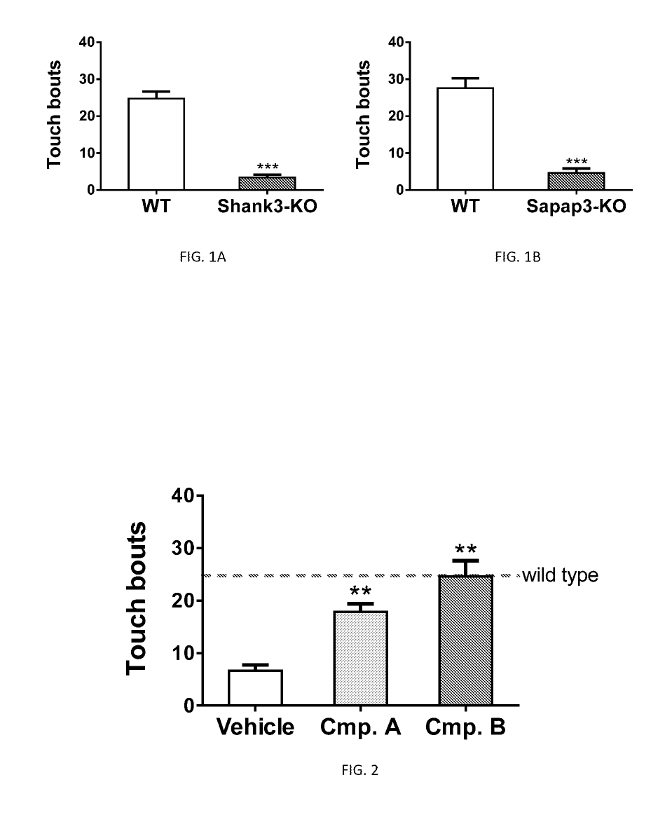

In addition to excessive grooming, both SAPAP3 and Shank3 knock-out mice show excessive anxiety, especially toward novel objects or animals (Peca et al., 2011, Nature, 472(7344):437-42; Welch et al., Nature 448:894, 2007). In order to determine whether a given compound can address these deficits, the present invention provides the Novelty Phobia test, which involves measuring the number of times mice interact with novel objects placed in their environment. Both Shank3 and SAPAP3 knock-out mice show significant deficits in this test compared to wild type littermates (see FIG. 1). This demonstrates that the increased anxiety observed in these mice also generates deficits similar to those seen in human conditions such as ASD and anxiety disorders (e.g., OCD).

### Behavioral Animal Model Drug Discovery Paradigm for ASD and Anxiety Disorders

The identification of compounds that are useful for treating human behavioral disorders, including ASD and anxiety disorders (e.g., OCD) is greatly facilitated by preclinical testing with appropriate animal models. In some aspects, the present invention encompasses the insight that the source of a problem encountered with other efforts to identify useful agents for the treatment of ASDs and/or anxiety disorders is the use of a limited set of models for characterizing such agents. Among other things, the present disclosure demonstrates that the described compounds induce a particular activity spectrum in a combination of transgenic and wild type in vivo model test systems that quantitatively measure behaviors relevant to ASD and various human anxiety disorders (e.g., OCD). This strategy includes use of genetic models of ASD and anxiety (e.g., OCD), namely, Shank3 and Sapap3, that have strong face and construct validity for their respective human diseases and show a clear component of anxiety. The complete evaluation of relevant behaviors in these animals is further augmented by the unique novelty phobia test provided herein that simultaneously measures fear of novelty and innate responses to stressful, anxiety-producing situations. Thus, among other things, the present disclosure defines and provides effective strategies for the identification and/or characterization of useful agents for the treatment of ASDs and/or anxiety disorders. In some embodiments, such strategies include combinations of assays, for example both genetic and situational models.

In some embodiments of the present invention, behavioral testing in genetic models is supplemented with additional tests using more standard rodent models of psychiatric disorders. Such tests have proven valuable for psychiatric drug discovery efforts targeted at treating behavioral deficits associated with a variety of psychiatric conditions, including those prominent in ASD and anxiety disorders (e.g., OCD). Such symptoms, including heightened anxiety, repetitive behaviors and social withdrawal or dysfunction, can be observed in certain wild type animal behavior test paradigms. However, this simple wild type animal behavioral model approach has recognized limitations in predicting human behaviors and drug efficacy in psychiatric disease. (Moser, Psychopharmcol. (Berl.) 99(1): 48, 1989; Silva and Brandao Pharm. Biochem. Behay. 65(2): 209, 2000; Lucki et al, Psychopharm. 155:315, 200; Griebel and Holmes, Nat. Rev. Drug. Disc. 12:667, 2013). Therefore, it is useful to assess a compound in multiple animal models of behavioral dysfunction to fully establish potential efficacy in human psychiatric disorders. Employing these wild-type animal models in combination with the disease-focused genetic models provided herein mitigates the recognized limitations of the former in predicting drug efficacy in human psychiatric disease.

One standard model of anxiety and repetitive behavior is the marble burying test (MBT) (Broekkamp et al Eur. J. Pharmacol., 126, 223-229, 1986), often implemented as an acute single-administration experiment. Compounds that are active or efficacious in the MBT reduce the propensity of the mouse to bury marbles placed in its environment as anxiety-generating foreign objects. Since the mice spend much of the test period burying marbles, and in some cases reburying them, the test may also be a measure of repetitive behavior. As a result the MBT may predict efficacy in treating OCD patients as repetitive behavior is a central component of the disease. For example, selective serotonin reuptake inhibitors (SSRIs) approved to treat OCD have been found to be efficacious after acute administration in this test (Njung'e and Handley, Br. J. Pharmacol. 104:105, 1991). This result is noteworthy as SSRI drugs demonstrate efficacy in human depression and OCD patients only after chronic administration (typically 2-3 weeks).

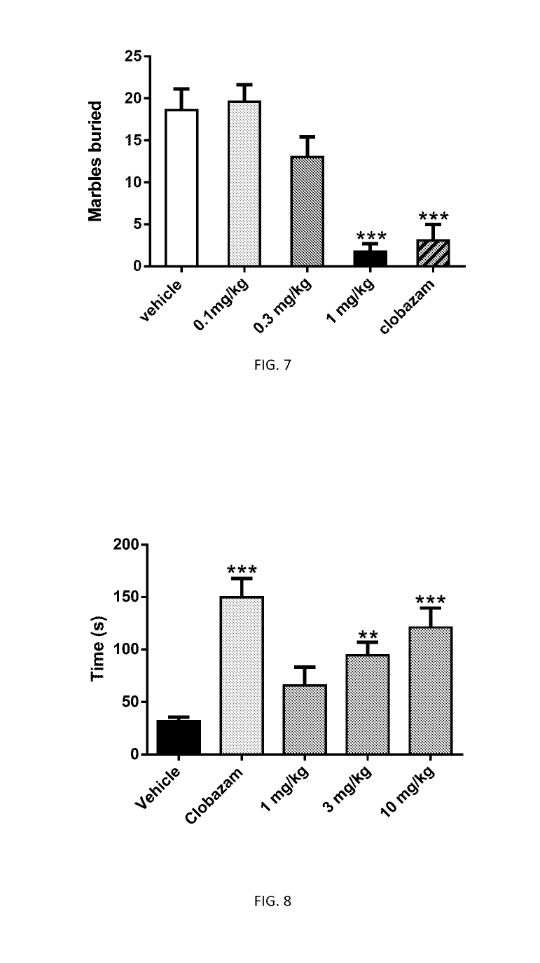

There is a single report in the literature of a positive effect of an NR2B antagonist in the marble burying test. The NR2B antagonist, Ro25-6981, showed modest efficacy after a single dose subcutaneous administration (lijima et al. Neuroscience, 2010, 471, 63-65) at the highest dose tested. However, the study lacked a positive control arm, rendering interpretation of this effect incomplete. In contrast, as described herein, it has been discovered that the potent and selective NR2B antagonist (−)-cis-4-methylbenzyl 3-fluoro-4-[(pyrimidin-2-ylamino)methyl]piperidine-1-carboxylate (Compound D) is highly effective in the marble burying test (see FIG. 7).

Another model of anxiety is the elevated plus maze test, most often conducted as an acute study in genetically normal rats or mice (Handley and Mithani, Naunyn. Schmied, Arch. Pharmacol., 327(1):1, 198). This test has been used routinely to identify and characterize candidate compounds that subsequently showed efficacy in human anxiety disorders. This is particularly true of human anxiolytic drugs from the benzodiazepine compound class after acute administration in the elevated plus maze test (Lister, Psychopharmacol. (Berl.) 92(2):180, 1987). Notably, the NR2B antagonist Ro25-6981 was shown to be inactive in the elevated plus maze test (Mathur et al., Pharmacol, Biochem., Behay. 91:453, 2008) in the only report yet found regarding NR2B antagonists in elevated plus maze. However, as described herein, it has been discovered that the potent and selective NR2B antagonist (−)-cis-4-methylbenzyl 3-fluoro-4-[(pyrimidin-2-ylamino)methyl]piperidine-1-carboxylate (Compound D) is highly effective in the elevated plus maze test (see FIG. 8).

### Transgenic+Behavioral Animal Models Drug Discovery Paradigm for ASD and Anxiety Disorders

As noted above, the present disclosure provides, among other things, the insight that many wild type animal behavioral models are significantly limited and/or ineffective in predicting human behaviors and drug efficacy in psychiatric disease. Provided herein are disease-focused genetic models that mitigate these limitations. The predictive value of any single test in this battery is compromised by theoretical and empirical shortcomings. However, compounds that are robustly active across a range in this series of tests should be effective in treating the analogous constellation of symptoms that are routinely present in human ASD and anxiety (e.g., OCD) patients.

### Transgenic Models and Novelty Phobia Test

Novelty phobia is a key feature of anxiety (e.g., OCD) and ASD in human patients, and this phenotype can be quantified in a mouse behavioral assay. In this behavioral assay, when a novel object was introduced to mouse home cage or assay chamber, wild type mice would normally show curiosity and check it out by touching the objects with their nose for multiple times. In contrast, Shank3-KO (ASD model) or Sapap3-KO (OCD model) mice displayed apparent phobia and stayed away from the novel object. By counting the number of bouts of the animals touching the object, the novelty phobia behavior of Shank3-KO or Sapap3-KO mice was quantitatively measured (Example 2.2). Shank3 and Sapap3 knock-out mice interacted with the novel object less frequently (endpoint=touch bouts) than did their wild type littermates (see FIG. 1). The wild type mice touched the novel object an average of approximately 25 times (n=8-10), whereas Shank3-KO mice touched the object only 1 to 8 times (average=4 times, n=10). Sapap3 knock-out mice showed a similar deficit. The novelty phobia behavior of Shank3-KO mice and sapap3-Ko does not change with age when evaluated between the ages of 4 weeks old and 16 weeks old.

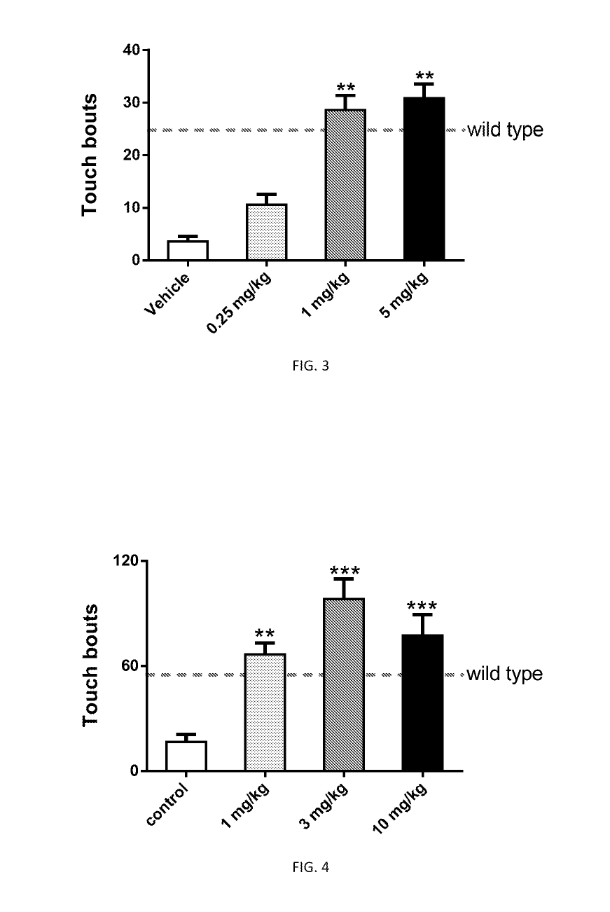

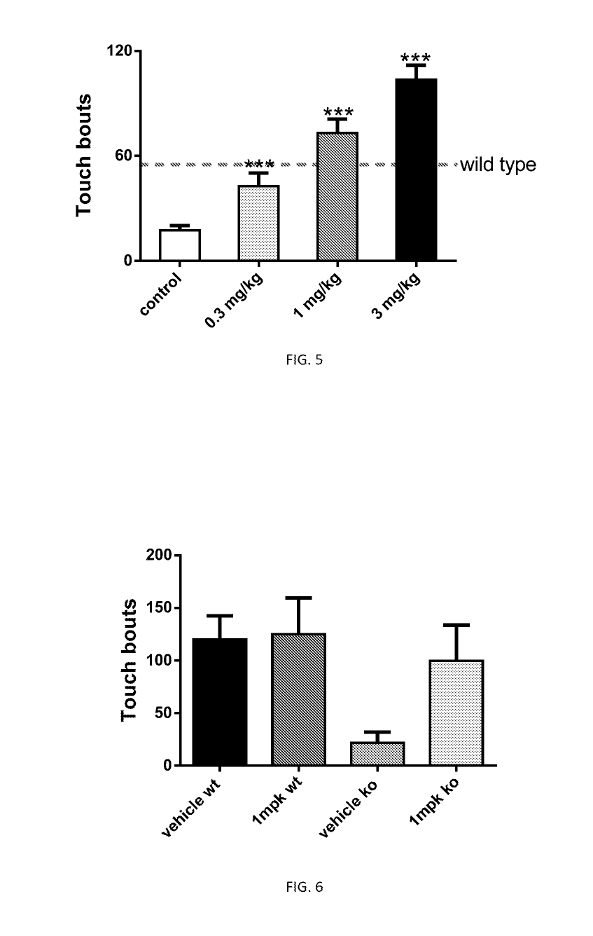

Administration of a six daily i.p. doses of two different NR2B subunit-selective NMDA antagonists, Compounds A and B, significantly reduced the novelty phobia observed in Shank3 knock-out mice (see FIG. 2). The mice received doses of 5 mg/kg once a day and were tested 30 minutes after the last dosing. This effect seen at relatively low doses of test compound is consistent with a selective effect at NR2B-NMDA receptors. The attenuation of novelty phobia by Compound B in Shank3 knock-out mice was dose-dependent and could be observed after a single, acute, i.p. dose (see FIG. 3). The effects of Compound B and Compound C on Sapap3 knock-out mice were nearly identical to the effect of NR2B antagonists on Shank3 knock-out mice (see FIGS. 4 and 5, respectively). Finally, FIG. 6 shows the results of single acute dose study demonstrating a robust effect of Compound D in the SAPAP3 novelty phobia test. Together these results demonstrate the utility of these NR2B subunit-selective NMDA receptor antagonists at reversing anxiety and phobic behaviors in two different genetic models that mimic the biochemical and behavioral pathology of humans suffering from anxiety or ASD.

### Wild Type Mouse Marble Burying Test

In addition to measurement of novelty phobia, the anxiolytic/OCD effects of Compound D were measured using the Marble Burying Test (Example 2.2). Compound D dose-dependently reduced the number of marbles buried during the test 20 minutes after an i.p. dose (see FIG. 7).

### Wild Type Mouse Elevated Plus Maze Test

Compound D showed a similar anxiolytic effect in the mouse Elevated Plus Maze (Example 2.2). Single i.p. doses of Compound D significantly increased the amount of time spent in the open arms of the maze, and the effect was again dose-dependent with a maximal efficacy approaching that of the benzodiazepine control compound clobazam (see FIG. 8). These results show that Compound D has significant anxiolytic activity and effects improvement in repetitive behavior, evidencing its utility in treating anxiety (e.g., OCD).

### EXAMPLES

### Example 1

### Synthesis of NR2B Subunit Selective NMDA Receptor Antagonists

Abbreviations:
aq aqueous
Boc t-butoxycarbonyl
Cbz benzyloxycarbonyl
DCM dichloromethane
DCE 1,2-dichloroethane
DIPEA N,N-diisopropylethylamine
DMF N,N-dimethylformamide
DMSO dimethyl sulfoxide
Et₂O diethyl ether (“ether”)
EtOAc ethyl acetate
EtOH ethanol
eq equivalents
h hours
HPLC high performance liquid chromatography
LC liquid chromatography
Me methyl
MS mass spectrometry
MS (ESI) mass spectrometry electrospray ionization
NMP N-methyl-2-pyrrolidone
NMR nuclear magnetic resonance
rt room temperature
Tf triflate
Tf₂O triflic anhydride
TFAA trifluoroacetic anhydride
THF tetrahydrofuran
TLC thin layer chromatography

### Example 1.1

### (+)-(1S,2S)-1-(4-hydroxyphenyl)-2-(4-hydroxy-4-phenylpiperidino)-1-propanol (Compound A)

(+)-(1S,2S)-1-(4-hydroxyphenyl)-2-(4-hydroxy-4-phenylpiperidino)-1-propanol (PubChem: CID 219101) was synthesized according to synthetic methods described and referred to in B. L. Chenard et al., J. Med. Chem. 1995; 38:3138-45.

### Example 1.2

### N-(1-(2,2-difluoro-2-(4-trifluoromethyl)phenyl-ethyl)piperidin-4-yl)-1H-pyrazolo[3,4-d]pyrimidin-4-amine (Compound B)

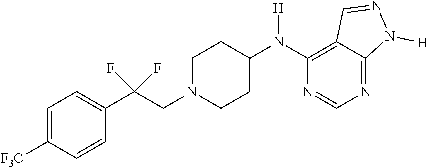 [SMILES: FC(F)(F)c1ccc(C(F)(F)CN2CCC(Nc3ncnc4[nH]ncc34)CC2)cc1]

### Step 1. ethyl 2,2-difluoro-2-(4-(trifluoromethyl)phenyl)acetate

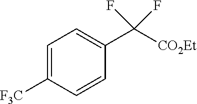 [SMILES: CCOC(=O)C(F)(F)c1ccc(C(F)(F)F)cc1]

A mixture of 1-iodo-4-(trifluoromethyl)benzene (10 g, 36 mmol), ethyl 2-bromo-2,2-difluoro-acetate (7.5 g, 36 mmol) and copper powder (4.60 g, 72 mmol) in DMSO (120 mL) was heated to 80° C. After stirring for 20 hrs at 80° C., the mixture was cooled down to rt and diluted with EtOAc. The mixture thus obtained was poured into the water and stirred for 0.5 h. The suspension was filtered through a pad of celite, and the filter mass was washed with EtOAc. The combined organic phases were washed with water and brine, dried over Na₂SO₄ and concentrated. The concentrate was purified by column chromatography over silica gel (100% hexane) to afford the title compound as pale brown oil (6.35 g, 64%). MS (ESI) calcd for C₁₁H₉F₅O₂: 268.1; found: [M+H]. ¹H NMR (400 MHz, CDCl₃) δ 7.78-7.70 (m, 4H), 4.31 (q, J=7.1 Hz, 2H), 1.31 (t, J=7.1 Hz, 3H).

### Step 2. 2,2-difluoro-2-(4-(trifluoromethyl)phenyl)ethanol

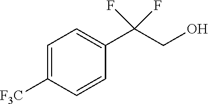 [SMILES: OCC(F)(F)c1ccc(C(F)(F)F)cc1]

To a suspension of LiBH₄ (3.25 g, 15 mmol) in dried THF (35 mL) was added a solution of ethyl 2,2-difluoro-2-(4-(trifluoromethyl)phenyl)acetate (2.0 g, 7.5 mmol) in THF, dropwise at 0° C. The mixture thus obtained was stirred for 30min at room temperature. After the ester was consumed, the reaction was quenched with 1M aqueous HCl at 0° C., and the mixture was extracted with EtOAc. The organic phases were washed with brine, dried over Na₂SO₄ and concentrated to afford the title compound as brown liquid (1.68 g, 100%). MS (ESI) calcd for C₉H₇F₅O: 226.1; found: [M+H]. ¹H NMR (400 MHz, CDCl₃) δ 7.75-7.64 (m, 4H), 4.00 (t, J=13.0 Hz, 2H).

### Step 3. 2,2-difluoro-2-(4-(trifluoromethyl)phenyl)ethyltrifluoromethanesulfonate

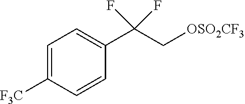

To a stirred solution of 2,2-difluoro-2-(4-(trifluoromethyl)phenyl)ethanol (140 mg, 0.67 mmol) and DIPEA (0.3 mL, 1.7 mmol) in dried ether (5 mL) was added Tf₂O (0.3 mL, 1.5 mmol) dropwise at 0° C. under N₂ atmosphere. After stirring for 30min at 0° C., the suspension was stirred an additional 1h at ambient temperature. After the alcohol was consumed, the suspension was filtered through a pad of Celite. The filtrate was concentrated and purified by column chromatography over silica gel (100% hexane) to afford the title compound as a colorless oil (220 mg, 52%). ¹H NMR (400 MHz, CDCl₃) δ 7.78 (d, J=8.2 Hz, 2H), 7.67 (d, J=8.2 Hz, 2H), 4.72 (t, J=11.6 Hz, 2H).

### Step 4. tert-butyl 1-(2,2-difluoro-2-(4-(trifluoromethyl)phenyl)ethyl)piperidin-4-yl-carbamate

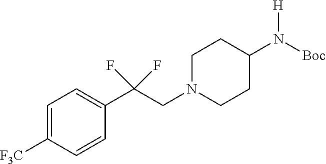 [SMILES: CC(C)(C)OC(=O)NC1CCN(CC(F)(F)c2ccc(C(F)(F)F)cc2)CC1]

A solution of 2,2-difluoro-2-(4-(trifluoromethyl)phenyl)ethyltrifluoromethanesulfonate (220 mg, 0.61 mmol), tert-butyl piperidin-4-ylcarbamate (360 mg, 1.82 mmol) and DIPEA (0.15 mL, 0.91 mmol) in DCM (5 mL) was heated to 40° C. After stirring overnight at 40° C., the solution was concentrated and purified by column chromatography over silica gel (eluent: 10% of EtOAc in hexane) to afford the title compound as off-white powder (200 mg, 80%). MS (ESI) calcd for C₁₃H₂₅F₅N₂O₂: 408.2; found: [M+H]. ¹H NMR (400 MHz, CDCl₃) δ 7.67 (d, J=8.4 Hz, 2H), 7.62 (d, J=8.4 Hz, 2H), 4.43-4.33 (m, 1H), 3.47-3.31 (m, 1H), 2.94 (t, J=13.8 Hz, 2H), 2.77-2.69 (m, 2H), 2.40-2.31 (m, 2H), 1.87-1.78 (m, 2H), 1.59 (s, 1H), 1.43 (s, 9H), 1.38-1.26 (m, 2H).

### Step 5. 1-(2,2-difluoro-2-(4-(trifluoromethyl)phenyl)ethyl)pipendin-4-amine

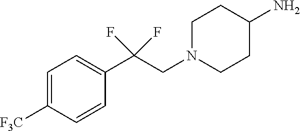 [SMILES: NC1CCN(CC(F)(F)c2ccc(C(F)(F)F)cc2)CC1]

To a stirred solution of tert-butyl 1-(2,2-difluoro-2-(4-(trifluoromethyl)phenyl)ethyl)piperidin-4-ylcarbamate (200 mg, 0.49 mmol) in DCM (5 mL) was added TFA (5 mL). After stirring for 30min, the mixture was concentrated, and the residue was dissolved in ice-water. The aqueous solution was basified with 1M aqueous NaOH. The aqueous phase was extracted with EtOAc. The combined organic phases were washed with brine, dried over Na₂SO₄ and concentrated to afford the amine as off-white powder (140 mg, 93%). MS (ESI) calcd for C₁₄H₁₇F₅N₂: 308.2; found: 309.2 [M+H]. ¹H NMR (400 MHz, CDCl₃) δ 7.70-7.60 (m, 4H), 2.94 (t, J=13.8 Hz, 2H), 2.79-2.70 (m, 2H), 2.66-2.57 (m, 1H), 2.35-2.24 (m, 2H), 1.73-1.66 (m, 2H), 1.62 (s, 2H), 1.35-1.23 (m, 2H).

### Step 6. N-(1-(2,2-difluoro-2-(4-(trifluoromethyl)phenyl)ethyl)pipendin-4-yl)-1H-pyrazolo-[3,4-d]pyrimidin-4-amine

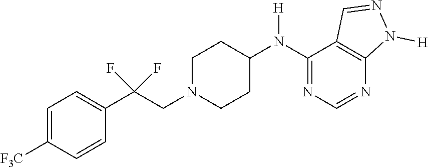 [SMILES: FC(F)(F)c1ccc(C(F)(F)CN2CCC(Nc3ncnc4[nH]ncc34)CC2)cc1]

To a stirred solution of 1-(2,2-difluoro-2-(4-(trifluoromethyl)phenyl)ethyl)-piperidin-4-amine (200 mg, 0.65 mmol) in 1-butanol (5 mL) was added 4-chloro-1H-pyrazolo[3,4-d]pyrimidine (100 mg, 0.65 mmol) and DIPEA (5.0 mL). The mixture was heated at 110° C. for 12 hours. The reaction mixture was concentrated under reduced pressure. The resulting residue was diluted with ethyl acetate (15 mL) and the organic phase was washed with saturated sodium bicarbonate (10 mL×3) and brine (10 mL×1), dried over anhydrous Na₂SO₄ and concentrated under reduced pressure. The residue was purified by preparative TLC (eluent: DCM/MeOH=10/1) to afford the title compound as an off-white powder (142 mg, 51%). MS (ESI) calcd for C₁₉H₁₉F₅N₆: 426.2; found: 427.4 [M+H]. ¹H NMR (400 MHz, CDCl₃) δ 8.37 (s, 1H), 7.96 (s, 1H), 7.70 (d, J=8.4 Hz, 2H), 7.66 (d, J=8.4 Hz, 2H), 3.04 (t, J=14.0 Hz, 2H), 2.88-2.91 (m, 2H), 2.52-2.59 (m, 2H), 2.05-2.08 (m, 2H), 1.54-1.74 (m, 2H).

N-(1-(2,2-difluoro-2-(4-trifluoromethyl)phenyl-ethyl)piperidin-4-yl)-1H-pyrazolo[3,4-d]pyrimidin-4-amine can also be prepared by any of the synthetic methods described and referred to in Intl. Patent Appl. Publ. No. WO 2006/113471 (the applicable portion of which is incorporated herein by reference).

### Example 1.3

### (±)-cis-4-methylbenzyl 3-fluoro-4-[(pyrimidin-2-ylamino)methyl]piperidine-1-carboxylate (Compound C)

(±)-cis-4-Methylbenzyl 3-fluoro-4-[(pyrimidin-2-ylamino)methyl]piperidine-1-carboxylate was synthesized according to the procedure described in WO 2004/108705 as its methanesulfonate salt (the applicable portion of which is incorporated by reference herein).

The individual (−) and (+) enantiomers can be obtained by chiral chromatography of the racemate as referred to in WO 2004/108705. Alternatively, each of the enantiomers can be stereoselectively synthesized using asymmetric hydrogenation methodology described in Intl. Patent Appl. Publ. No. WO 2006/069287 (the applicable portion of which is incorporated by reference herein) and in S. W. Krska et al., Tetrahedron, 2009, 65, 8987-8994 (the entirety of which is incorporated by reference herein).

### Example 1.4

### (−)-(3S,4R)-cis-4-methylbenzyl 3-fluoro-4-[(pyrimidin-2-ylamino)methyl]-piperidine-1-carboxylate (Compound D)

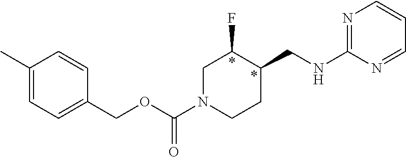 [SMILES: Cc1ccc(COC(=O)N2CC[C@H](CNc3ncccn3)[C@H](F)C2)cc1]

### Step 1. (3-fluoropyridin-4-yl)methanol

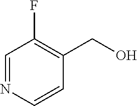

A mechanically stirred solution of 3-fluoropyridine (600 g, 6.18 mol) in dry THF (6 L) was cooled to −65° C. under N₂ atmosphere. Lithium diisopropylamine solution in THF (3.4 L, 6.5 mol) was added over 2 hours while keeping the temperature below −60° C., and the reaction mixture was allowed to stir an additional hour below −60° C. Neat DMF (804 mL, 10.4 mol) was then added over a one hour period at a rate as to maintain the temperature below −60° C. The reaction mixture was monitored by TLC for the complete consumption of 3-fluoropyridine starting material. Upon completion, the reaction mixture was then warmed to −10° C., and H₂O (1.1 Kg, 62 mol) was added. Sodium borohydride (234 g, 6.18 mol) was then added in two portions over several minutes at 0° C. with stirring. Then 6 M HCl (5.6 L) was added over 1 h while maintaining the reaction quenching temperature between 0-25° C. The reaction mixture was then heated to 40° C. and stirred at this temperature overnight. To the mixture 6 M NaOH was then slowly added at 0-15° C. to adjust the pH to 12. The aqueous layer was extracted with isopropylacetate (500 mL×1, 1 L×3). The combined organic layers were washed with brine, dried over Na₂SO₄ and concentrated in vacuo. To the crude product was added heptane-isopropylacetate (1.2 L, v/v=5/1), and the mixture was stirred at 0° C. for 30 min. The product crystallized as a pale-yellow solid during stirring. The mixture was filtered, the filter cake was extracted with cooled heptane (250 mL×1), collected and dried under vacuum to give the title compound as a pale-yellow solid (688 g, 88%). ¹H NMR (400 MHz, CDCl₃) δ 8.42 (d, J=4.8 Hz, 1H), 8.40 (d, J=1.6 Hz, 1H), 7.49 (t, J=4.8 Hz, 1H), 4.85 (s, 2H), 2.35 (br s, 1H).

### Step 2. 1-benzyl-3-fluoro-4-(hydroxymethyl)pyridin-1-ium bromide

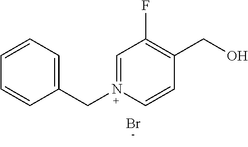

To a stirred solution of (3-fluoropyridin-4-yl)methanol(688 g, 5.40 mol) in acetone (6.8 L) was added neat BnBr (1018 g, 5.90 mol). The resulting mixture was stirred overnight at reflux. The reaction mixture was cooled to room temperature and diluted with heptane (1 L). The suspension was filtered, and the wet cake was then stirred with 25% acetone/heptane v/v (3 L) and filtered. The filter mass was then dried in vacuo to afford the title compound as pale-yellow solid (1570 g, 97%).

¹H NMR (400 MHz, CD₃OD) δ 8.97 (d, J=6.4 Hz, 1H), 8.28 (t, J=6.4 Hz, 1H), 7.57-7.52 (m, 2H), 7.53-7.48 (m, 3H), 5.85 (s, 2H), 4.96 (s, 2H).

### Step 3. (1-benzyl-5-fluoro-1,2,3,6-tetrahydropyridin-4-yl)methanol hydrochloride

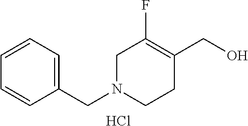 [SMILES: OCC1=C(F)CN(Cc2ccccc2)CC1 ; Cl]

To the stirred solution of 1-benzyl-3-fluoro-4-(hydroxymethyl)pyridin-1-ium bromide (1.55 Kg, 5.19 mol) in methanol (8.0 L) was added NaBH₄ (296 g, 7.82 mol) portionwise while maintaining the reaction temperature at ca. 0° C., followed by addition of water (16.0 L) while maintaining the reaction temperature below 0° C. The mixture was allowed to warm to room temperature. The aqueous layer was extracted with isopropylacetate (2.0 L×3), and the combined organic phases were dried and concentrated in vacuo. The residue was dissolved in 5% methanol in isopropylacetate (2.0 L). The solution was warmed to 50° C., followed by addition of a solution of 4 M HCl in dioxane (1.43 L). The resulting mixture allowed to room temperature, and stirred overnight. The precipitate was collected by filtration, washed with 5% methanol-isopropylacetate (2.0 L), heptane (1.5 L) and dried under vacuum. The crude product was dissolved in 50% methanol-isopropyl (14.0 L), and charcoal (255 g) was added. The resulting mixture was heated to reflux, and stirred for 2 hours. Then the suspension was filtered through celite. The filter cake was extracted with 20% methanol-isopropylacetate (6.0 L). The filtrate was collected and concentrated to roughly 25% of its original volume and the solid precipitate was isolated by filtration. The solid was extracted with 5% methanol-isopropylacetate (4.0 L), and dried overnight under vacuum to afford the title compound as an off-white solid (1.08 Kg, 81%).¹H NMR (400 MHz, CD₃OD) δ 7.61-7.56 (m, 2H), 7.55-7.52 (m, 3H), 4.47 (br s, 2H), 4.21 (br s, 2H), 3.84 (br s, 2H), 3.36 (br s, 2H), 2.59 (br s, 2H).

### Step 4. ((3S,4R)-1-benzyl-3-fluoropiperdin-4-yl)methanol

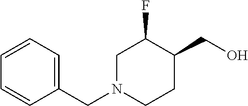 [SMILES: OC[C@H]1CCN(Cc2ccccc2)C[C@H]1F]

Under N₂ atmosphere, a three-necked round-bottom flask was charged with 180 mg of (R)-(+1- [(R)-2-(2′-(diphenylphosphino)phenyl]ferrocenylethyldicyclohexylphosphine (CA number 388079-60-5, available from Solvias as Walphos # SL-W003-1), 60 mg of cycloctadiene rhodium chloride dimer [(COD)RhCl]₂ and dry nitrogen degassed dichloromethane (10 mL). The solution was stirred at ambient temperature for 45 min. A solution of (1-benzyl-5-fluoro-1,2,3,6-tetrahydropyridin-4-yl)methanol hydrochloride (40.0 g, 155 mmol) in dry MeOH (130 mL, N₂ degassed) was added to a nitrogen purged 250 mL stainless steel pressure vessel. Subsequently, the aged catalyst solution from above was added into the vessel under nitrogen flow. The resulting mixture was degassed three times with H₂ and then (2.0 MPa, ca. 300 psi) heated at 50° C. under 2.0 MPa (ca. 300 psi) of H₂ for 3 days with stirring. After the reaction was deemed complete by HPLC analysis, the reaction vessel allowed to cool to room temperature and was purged with nitrogen. The reaction mixture was concentrated in vacuo to yield a dark brown oil. This concentrate was taken up in EtOAc and saturated aqueous NaHCO₃ was added. The mixture was stirred at room temperature for half hour and the organic phase was separated. The aqueous phase was extracted with three times with EtOAc. The combined organic phases were washed with brine, dried over Na₂SO₄, and concentrated under vacuum. The residue was purified by column chromatography over silica gel to (eluent: 50% EtOAc in hexane) afford the title compound as a yellow oil (27.4 g, 73%). ¹H NMR (400 MHz, CDCl₃) δ7.33-7.27 (m, 4H), 7.27-7.23 (m, 1H), 4.83 (d, J=48.8 Hz,1H), 3.77-3.72 (m, 1H), 3.64-3.52 (m, 3H), 3.24-3.18 (m,1H), 2.97-2.92 (m, 1H), 2.16-2.04 (m, 2H), 1.77-1.65 (m, 3H).

### Step 5. ((3S,4R)-3-fluoropiperidin-4-yl)methanol hydrochloride

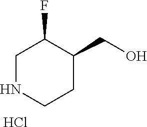

To a stirred solution of ((3S,4R)-1-benzyl-3-fluoropiperdin-4-yl)methanol (24.0 g, 108 mmol) in MeOH (100 mL) was added 10% Pd/C (2.4 g,) and 6M aqueous HCl (18 mL). The mixture was purged with hydrogen three times, and hydrogenated at atmospheric pressure. After stirring at room temperature for 6 hours, the starting material was consumed. The reaction vessel was purged with nitrogen. The reaction mixture then was filtered through celite, the filter pad rinsed with MeOH, and the filtrate concentrated in vacuo. The residue obtained was dissolved in a solution of methanol/isopropylacetate (v/v=1/1), and the mixture was filtered. The filtrate was concentrated and then dissolved in 10% methanol/isopropylacetate. The title compound crystallized from the solution as an off-white solid. The solid was isolated by filtration, washed twice with 10% methanol/isopropylacetate and dried under vacuum to afford the title compound as an off-white solid (11.2 g, 62%). ¹H NMR (400 MHz, CD₃OD) 6 5.10 (d, J=46.0 Hz, 1H), 3.69-3.58 (m, 2H), 3.52 (dd, J=10.8 and 6.4 Hz, 1H), 3.44-3.37 (m, 1H), 3.28 (dd, J=40 and 14 Hz, 1H), 3.13-3.06 (m, 1H), 2.08-1.89 (m, 1H), 1.88-1.82 (m, 1H), 1.77-1.63 (m, 1H).

### Step 6. (3S,4R)-4-methylbenzyl 3-fluoro-4-(hydroxymethyl)piperidine-1-carboxylate

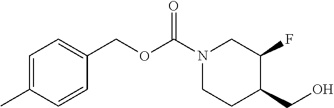 [SMILES: Cc1ccc(COC(=O)N2CC[C@H](CO)[C@H](F)C2)cc1]

To a stirred solution of 4-methylbenzyl alcohol (36.0 g, 296 mmol) in dimethylformamide (300 mL) was added N,N′-carbonyldiimidazole (48.0 g, 296 mmol). The mixture was stirred at room temperature for one hour and the solid ((3S,4R)-3-fluoropiperidin-4-yl)methanol hydrochloride (50 g, 296 mmol) was added portionwise. This reaction mixture was stirred at 50° C. overnight and allowed to cool to room temperature. The solvent was removed under reduced pressure and the residue was dissolved in ethylacetate. The solution was washed with 0.5M aqueous HCl, saturated NaHCO₃, brine, dried over Na₂SO₄ and concentrated under reduce pressure. The residue was purified by column chromatography over silica gel (50% ethylacetate-hexane) to afford the title compound as colorless oil, which gradually became a white solid on standing (65.0 g, 78%). ¹H NMR (400 MHz, CDCl₃) δ 7.25 (d, J=8.0 Hz, 2H), 7.17 (d, J=8.0 Hz, 2H), 5.09 (s, 2H), 4.83-4.46 (m, 2H), 4.37-4.22 (m, 1H), 3.46-3.40 (m, 1H), 3.32-3.27 (m, 1H), 3.00-2.72 (m, 2H), 2.35 (s, 3H), 1.86-1.70 (m, 1H), 1.67-1.54 (m, 2H).

### Step 7. (3S,4R)-4-methylbenzyl 3-fluoro-4-((methylsulfonyloxy)methyl)piperidine-1-carboxylate

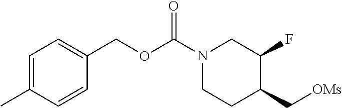 [SMILES: Cc1ccc(COC(=O)N2CC[C@H](COS(C)(=O)=O)[C@H](F)C2)cc1]

To a stirred solution of ((3S,4R)-4-methylbenzyl 3-fluoro-4-(hydroxymethyl)-piperidine-1-carboxylate (55.0 g, 196 mmol) and diisopropylethylamine (80 mL, 455 mmol) in dichloromethane (500 mL) was added neat methanesulfonylchloride (25.2 mL, 325 mmol) dropwise at ice-water bath temperature under N₂. The mixture was allowed to warm to room temperature and was stirred for 30 min. The mixture was diluted with dichloromethane and water was added. The aqueous layer was extracted with dichloromethane and the combined organic layers were dried over Na₂SO₄ and concentrated in vacuo. The residue was purified by column chromatography over silica gel (30% ethylacetate:hexane) to afford the title compound as a white powder (59.0 g, 84%). ¹H NMR (400 MHz, CDCl₃) δ 7.25 (d, J=8.0 Hz, 2H), 7.17 (d, J=8.0 Hz, 2H), 5.10 (s, 2H), 4.94-4.26 (m, 3H), 4.25-4.13 (m, 2H), 3.03 (s, 3H), 3.02-2.72 (m, 2H), 2.35 (s, 3H), 2.19-2.03 (m, 1H), 1.69-1.53 (m, 2H).

### Step 8. (3S,4R)-4-methylbenzyl 4-(azidomethyl)-3-fluoropiperidine-1-carboxylate

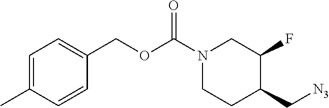 [SMILES: Cc1ccc(COC(=O)N2CC[C@H](CN=[N+]=[N-])[C@H](F)C2)cc1]

To a stirred solution of (3S,4R)-4-methylbenzyl 3-fluoro-4-((methylsulfonyloxy)methyl) piperidine-1- carboxylate (59.0 g, 164 mmol) in dimethylformamide (200 mL) was added sodium azide (21.3 g, 328 mmol) at room temperature. The mixture was heated at 90° C. overnight and then allowed to cool to room temperature. The reaction mixture was diluted with ethylacetate, followed by addition of water. The aqueous layer was separated and extracted with ethylacetate. The combined organic layers were dried over Na₂SO₄ and concentrated in vacuo. The residue was purified by column chromatography over silica gel (20% ethylacetate:hexane) to afford the title product as a colorless oil (50.2 g, 92%).¹H NMR (400 MHz, CDCl₃) δ 7.25 (d, J=8.0 Hz, 2H), 7.17 (d, J=8.0 Hz, 2H), 5.10 (s, 2H), 4.84-4.16 (m, 3H), 3.45-3.40 (m, 1H), 3.32-3.27 (m, 1H), 3.00-2.70 (m, 2H), 2.35 (s, 3H), 1.86-1.71 (m, 1H), 1.68-1.57 (m, 2H).

### Step 9. (3S,4R)-4-methylbenzyl 4-(aminomethyl)-3-fluoropiperidine-1-carboxylate hydrochloride

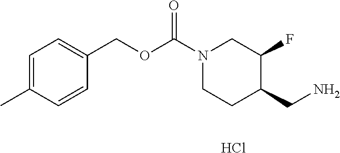 [SMILES: Cc1ccc(COC(=O)N2CC[C@H](CN)[C@H](F)C2)cc1 ; Cl]

To a stirred solution of (3S,4R)-4-methylbenzyl 4-(azidomethyl)-3-fluoropiperidine-1-carboxylate (50.0 g, 164 mmol) in MeOH (500 mL) was added SnCl₂.H₂O (83.8 g, 328 mmol). The mixture was stirred at room temperature overnight. The solvent was removed under reduced pressure and the residue was diluted with ethylacetate. The organic layer was washed with 10% NaOH, water, and brine. The organic phase was dried over Na₂SO₄ and concentrated to afford the crude (3S,4R)-4-methylbenzyl 4-(aminomethyl)-3-fluoropiperidine-1-carboxylate free base product as a yellow oil. This material was dissolved in toluene/MeOH (95/5, v/v) and HCl/Et₂O (82 mL, 2.0M, 164 mmol) solution was added at room temperature. The mixture was stirred at r.t. and the product crystallized from the solution as a white solid. The solid was collected by filtration, extracted twice with chilled toluene and dried under vacuum to afford the title compound as a white solid (33.0 g, 66%). ¹H NMR (400 MHz, CD₃OD) δ 7.24 (d, J=8.0 Hz, 2H), 7.16 (d, J=8.0 Hz, 2H), 5.08 (s, 2H), 4.86-4.67 (m, 1H), 4.51-4.45 (m, 1H), 4.27-4.23 (m, 1H), 3.21-2.87 (m, 4H), 2.35 (s, 3H), 2.19-1.97 (m, 1H), 1.70-1.53 (m, 2H). [α]_{D}=−31° (c=1.0, EtOH, 22° C.).

### Step 10. (−)-(3S,4R)-4-methylbenzyl 3-fluoro-4-((pyrimidin-2-ylamino)methyl)piperidine-1-carboxylate

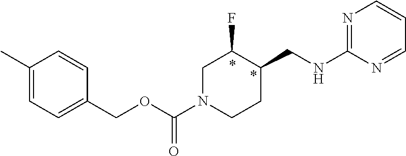 [SMILES: Cc1ccc(COC(=O)N2CC[C@H](CNc3ncccn3)[C@H](F)C2)cc1]

A mixture of (3S,4R)-4-methylbenzyl 4-(aminomethyl)-3-fluoropiperidine-1-carboxylate hydrochloride (1.20 g, 3.8 mmol), 2-chloropyrimidine (480 mg, 4.17 mmol) and diisopropylethylamine (2.0 mL, 11 mmol) in ethylene glycol (20 mL) was heated to 100° C. under nitrogen overnight. The mixture was cooled to room temperature and EtOAc and water were added. The EtOAc phase was washed with water and brine, dried over Na₂SO₄ and concentrated in vacuo. The residue was purified by column chromatography over silica gel (hexane/EtOAc=3/2) to afford the title compound as an off-white powder (1.05 g, 77%). MS (ESI) calcd for C₁₉H₂₃FN₄O₂: 358.2; found: 359.3 [M+H]. ¹H NMR (400 MHz, CDCl₃) 6 8.27 (d, J=4.8 Hz, 2H), 7.25 (d, J=8.0 Hz, 2H), 7.16 (d, J=8.0 Hz, 2H), 6.54 (t, J=4.8 Hz, 1H), 5.45 (s, 1H), 5.15-5.05 (m,2H), 4.90-4.18 (m, 3H), 3.52-3.39 (m, 2H), 2.97-2.72 (m, 2H), 2.35 (s, 3H), 2.12-1.88 (m, 1H), 1.63-1.56 (m, 2H). [α]_{D}=−43° (c=1.0, EtOH, t=22° C.).

Optical purity=95% ee, determined via chiral HPLC method (CHIRALPAK AD-H4.6*150 mm, 5 um, mobile phase: hexane/EtOH=65/35, flow rate=1.5 mL/min) with complete resolution of racemic cis-4-methylbenzyl 3-fluoro-4-((pyrimidin-2-ylamino)methyl)piperidine-1-carboxylate. Retention time of (−)-cis-(3S, 4R)-4-methylbenzyl 3-fluoro-4-((pyrimidin-2-ylamino)methyl)piperidine-1-carboxylate=7.97 min.

### Example 1.4a

### (−)-(3S,4R)-cis-4-methylbenzyl 3-fluoro-4-[(pyrimidin-2-ylamino)methyl]-piperidine-1-carboxylate methanesulfonate

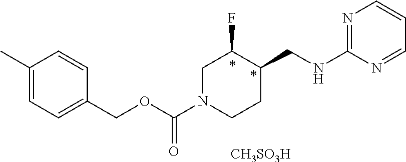

To a stirred solution of (−)-cis-(3S,4R)-4-methylbenzyl 3-fluoro-4-((pyrimidin-2-ylamino)methyl)-piperidine-1-carboxylate (1.05 g, 2.94 mmol) in dichloromethane/MeOH (20 mL/20 mL) was added 1.0M methanesulfonic acid in MeOH (3.1 mL, 3.1 mmol) at room temperature. After stirring for 30 min at rt, the clear solution was concentrated. The concentrate was washed with ether to afford the title compound as white powder (1.24 g, 92%). MS (ESI) calcd for C₁₉H₂₃FN₄O₂: 358.2; found: 359.3 [M+H]. ¹H NMR (400 MHz, CD₃OD) δ 8.84-8.42 (m, 2H), 7.24 (d, J=8.0 Hz, 2H), 7.16 (d, J=8.0 Hz, 2H), 7.02 (t, J=4.8 Hz, 1H), 5.07 (s, 2H), 4.86-4.69 (m, 1H), 4.49-4.42 (m, 1H), 4.26-4.22 (m, 1H), 3.64-3.50 (m, 2H), 3.17-2.82 (m, 2H), 2.72 (s, 3H), 2.33 (s, 3H), 2.22-2.05 (m, 1H), 1.66-1.57 (m, 2H).

(−)-cis-4-Methylbenzyl 3-fluoro-4-[(pyrimidin-2-ylamino)methyl]piperidine-1-carboxylate can also be prepared either by chiral chromatography according to WO 2004/108705 or prepared by asymmetric synthesis according to WO 2006/069287 and S. W. Krska et al., Tetrahedron, 2009, 65, 8987-8994.

### Example 1.5

### (+)-(3S,4R)-cis-4-methylbenzyl 3-fluoro-4-[(pyrimidin-2-ylamino)methyl]-piperidine-1-carboxylate

The title compound was prepared in analogous fashion to (−)-(3S,4R)-4-methylbenzyl 3-fluoro-4-((pyrimidin-2-ylamino)methyl)piperidine-1-carboxylate using the enantiomeric (S)-(−)-1-[(S)-2-(2′-(diphenylphosphino)phenyl]ferrocenylethyldicyclohexylphosphine (CA number 849925-19-5, available from Solvias as Walphos # SL-W003-2) as the chiral ligand in Step 4.

Optical purity 93% ee, determined via chiral HPLC method (CHIRALPAK AD-H4.6*150 mm, 5 um, mobile phase: hexane/EtOH=65/35, flow rate=1.5 mL/min) with complete resolution of racemic cis-4-methylbenzyl 3-fluoro-4-((pyrimidin-2-ylamino)methyl)piperidine-1-carboxylate. Retention time of (+)-cis-(3S,4R)-4-methylbenzyl 3-fluoro-4-((pyrimidin-2-ylamino)methyl)piperidine-1-carboxylate=9.58 min).

### Example 2

### Assays

### Example 2.1

### NR2B Antagonist Activity

HEK293 cell lines stably expressing cloned human NR1/NR2B and NR1/NR2A, respectively, were established according to standard previously described methods (Hansen et al., Comb. Chem High Throughput Screen. 11:304, 2008). Activation of the NR2A or NR2B subtype of NMDA receptor with glutamate as an agonist and glycine co-agonist on these cells results in calcium influx, which can be monitored with fluorescent indicator Fluo-4. A cell based assay has been implemented to evaluate the effect of a compound on NR2A and NR2B receptors by measuring the fluorescent changes (Hansen et al., Comb. Chem High Throughput Screen. 11:304, 2008).

HEK293 cells stably expressing NR2A or NR2B receptors were cultured at 37° C. in a humidified CO₂ incubator in DMEM supplemented with 10% fetal bovine serum (FBS) (Hyclone), 10 μM MK801 (Sigma-Aldrich) and 50 μM AP-5 (Tocris). For experiments, the cells were seeded onto poly-D-lysine-coated 96-well black plates with clear bottom (Corning) at a density of ˜50,000 cells/well. After overnight culture, the growth medium was removed from the wells and the cells were incubated at 37° C. for 60 min in Hanks buffer containing 4 μM fluo-4-AM (Invitrogen) and 0.1% bovine serum albumin (BSA). After dye-loading, the cells were washed three times with Hanks buffer and incubated for 10 min at room temperature with various concentrations of test compounds prepared in Hanks buffer with 0.1% BSA. The cell plates were placed onto FDSS μCell fluorescence reader (Hamamatsu). After 20 sec reading of background fluorescence, agonist glutamate at final 100 μM and co-agonist glycine at final 50 μM were added to the cells to activate the receptor, and the resulting fluorescence changes were recorded and quantified. Based on the changes in fluorescence intensity, the pharmacological effect of test compounds were analyzed and the IC₅₀ values derived from a non-linear least squares fitting of the concentration-dependent response to a standard logistic equation using Prism (Graphpad, Inc):

Amplitude=Max Amplitude/(1+(IC₅₀/[antagonist])°).

Results are shown in Table 2.1A, below.

TABLE 2.1A

| | | hNR2B IC₅₀ | hNR2A IC₅₀ |
|---|---|---|---|
| Cpd | Name | (nM) | (nM) |
| A | (+)-(1S,2S)-1-(4-hydroxyphenyl)-2- | 8-13 to 60^{‡} | 10,000^{‡} |
| | (4-hydroxy-4-phenyl-piperidino)-1- | | |
| | propanol | | |
| B | N-(1-(2,2-difluoro-2-(4-trifluoro- | 24 | >10,000 |
| | methyl)phenyl-ethyl)-piperidin-4- | | |
| | yl)-1H-pyrazolo[3,4-d]pyrimidin- | | |
| | 4-amine | | |
| D | (−)-cis-4-methylbenzyl 3-fluoro- | 11 | >10,000 |
| | 4-[(pyrimidin-2-ylamino)- | | |
| | methyl]piperidine-1-carboxylate | | |
| | (+)-cis-4-methylbenzyl 3-fluoro- | 18 | >10,000 |
| | 4-[(pyrimidin-2-ylamino)- | | |
| | methyl]piperidine-1-carboxylate | | |

^{‡}L. Mony et al., Br. J. Pharmacol. 2009; 157(8): 1301-17.

### Example 2.2

### Behavioral Assays

Animals. Shank3 knockout mice and Sapap3 knockout mice were purchased from Duke University. The animals were raised in a SPF environment according to AALAC guidelines. The breeding conditions and genotyping method were the same as described in J. M. Welch et al., Nature 2007; 448(7156):894-900, and in J. Peca et al., Nature 2011;472(7344):437-442. Homozygous knockout mice of 4 to 8 weeks of age were used in behavioral assays.

The test compounds were dissolved in a small volume of DMSO and diluted saline and administered intraperitoneally (i.p.) to 4 to 8 week old mice. About 30-60 minutes after dosing, the mice were subjected to a behavioral test as described below.

### Example 2.2.1

### Novelty Phobia Test

The behavioral testing apparatus was a rectangular black Plexiglas box, with no top or bottom (50 cm long, 26 cm wide and 15 cm tall). The chambers of the testing apparatus were cleaned and fresh paper chip bedding was added between tests. Behavioral tests were conducted between 10:00 am and 1:00 pm in dim light. Mice were brought to the experiment room in their home cages and administered intraperitoneally with drugs or vehicle in a volume of 10 ml/kg. About 30 to 60 min after drug administration, the mice were placed into the chamber and allowed to free play for 10 min. After this habituation period, a novel object (e.g., a toy ball of 3 cm in diameter with spikes) was introduced to the center of the chamber for 5 min. For the entire 15 min that the mouse spent in the chamber, video was recorded and analyzed with AnyMaze software (purchased from Stoelting Co.). The total distance traveled during the entire 15 min and the bouts that the mice touched the novel object during the last 5 min were measured. Statistical significance between two groups was determined by t test. In all statistical measures, the criterion for significance was set at p<0.05.

Mice lacking synaptic scaffolding proteins Shank3 and Sapap3 have marked novelty phobia. FIG. 1A shows the mean±SEM number of touch bouts measured during a 5-minute session with a novel object present in the test arena in wild type (WT) and Shank3 knock-out mice as indicated. Shank3 knock-out mice exhibited significantly fewer touch bouts than wild type mice (*** −p<0.001, ANOVA, n=8-10). FIG. 1B shows a similar experiment conducted on wild type (WT) and Sapap3 knock-out mice. Sapap3 knock-out mice had significantly fewer touch bouts than wild type mice (***−p<0.001, ANOVA, n=8-10).

Chronic, daily dosing Compounds A and B improves novelty phobia in Shank3 knock-out mice. FIG. 2 shows the mean±SEM number of touch bouts measured during a 5-minute session with a novel object present in the test area. The dashed line represents the average number of touch bouts observed in wild type mice for reference. One group of mice was treated i.p. once/day for 6 days with vehicle, a second group received daily i.p. injections of 5 mg/kg Compound A, and the third group received daily i.p. injections of 5 mg/kg Compound B. Testing was conducted 30-60 minutes after the last daily dose. Drug treatment significantly improved novelty phobia (**−p<0.05, ANOVA, n=8-10).

Compound B dose-dependently improved novelty phobia after a single administration in Shank3 knock-out mice. FIG. 3 shows the mean±SEM number of touch bouts measured during a 5-minute session with a novel object present in the test area. The dashed line represents the average number of touch bouts observed in wild type mice for reference. Groups of Shank3 knock-out mice were treated with vehicle or the indicated dose of Compound B by i.p. injection 30-60 minutes before testing. The groups treated with 1 and 5 mg/kg Compound B exhibited significantly improved novelty phobia compared to vehicle (**−p<0.05, ANOVA, n=8-10).

Compound B dose-dependently improved novelty phobia after a single administration in Sapap3 knock-out mice. FIG. 4 shows the mean±SEM number of touch bouts measured during a 10-minute session with a novel object present in the test area. The dashed line represents the average number of touch bouts observed in wild type mice for reference. Groups of Sapap3 knock-out mice were treated with vehicle or the indicated dose of Compound B by i.p. injection 30-60 minutes before testing. Novelty phobia was significantly improved by each test dose of Compound B compared to vehicle (**/***−p<0.05/0.001 respectively, ANOVA, n=8-10).

Compound C dose-dependently improved novelty phobia after a single administration in Sapap3 knock-out mice. FIG. 5 shows the mean ±SEM number of touch bouts measured during a 10-minute session with a novel object present in the test area. The dashed line represents the average number of touch bouts observed in wild type mice for reference. Groups of Sapap3 knock-out mice were treated with vehicle or the indicated dose of Compound C by i.p. injection 30 minutes before testing. Novelty phobia was significantly improved by all three test dose of Compound C compared to vehicle (***−p<0.001 respectively, ANOVA, n=8-10).

Compound D improved novelty phobia in Sapap3 knockout mice. FIG. 6 shows mean ±SEM number of touch bouts measured during a 10-minute session with the novel object present in the test arena. Groups of wild type and SAPAP3 knock-out mice were treated with vehicle or 1 mg/kg Compound D administered by i.p. injection 20 minutes prior to testing.

### Example 2.2.2

### Elevated Plus Maze

The Elevated Plus Maze, which detects anxiolytic activity, follows that described by S. L. Handley and S. Mithani Naunyn. Schmied. Arch. Pharmacol. 1984, 327, 1-5. Rodents avoid open spaces (the open arms of an elevated plus-maze). Anxiolytics increase exploratory activity in the open arms, as indicated by increased time spent on the open arms and/or by increased % open-arm entries. The maze consists of 4 arms of equal length and width (14×5 cm) arranged in the form of a plus sign (+). Two opposite arms are enclosed by 12 cm high walls (closed arms). The 2 other arms have no walls (open arms). The maze is raised approximately 60 cm above the floor. A mouse was placed in the center of the plus-maze and left to explore for 5 minutes. The number of entries into the open and closed arms and the time spent on the open arms were recorded. The percentage of open arm entries (open arm entries/total arm entries×100) was calculated. Ten mice were studied per group. The test was performed blind (test substances versus vehicle). Data with the test substances were analyzed by comparing treated groups (7 groups except for the reference substance) with vehicle control using one-way ANOVA followed by post-hoc Dunnett's tests. Data with the reference substance were analyzed using unpaired Student's t tests.

Compound D dose-dependently increased the amount of time spent by wild-type mice in the open arms of the elevated plus maze. FIG. 8 shows the mean ±SEM time spent in the open arms measured in mice treated with a single i.p. dose of test drug at the indicated dose 20 minutes before the test. Clobazam served as the positive control and was administered i.p. 30 minutes before testing at a dose of 8 mg/kg. The effect of 3 and 10 mg/kg Compound D and clobazam was significantly different from that of vehicle (**/*** p<0.01/0.001, ANOVA, n=10).

### Example 2.2.3

### Marble Burying Test

The Marble Burying Test was conducted as previously described by C. L. Broekkamp et al., Eur. J. Pharmacol. 1986, 126, 223-229. Mice exposed to novel object (marbles) will bury them in the sawdust floor covering. Anxiolytics decrease the number of marbles buried at non-sedative doses. Mice were individually placed in transparent plastic cages (33×21×18 cm) with 5 cm of sawdust on the floor 25 marbles grouped in the center of the cage. The cage was covered with an inverted plastic cage. Each test cage, together with the marbles, was impregnated with mouse odor before-hand by leaving 10 mice in the cage for 15 minutes. These mice then played no further role in the experiment. The number of marbles covered by sawdust (⅔ or more) was counted at the end of a 30-minute test. Twelve mice were studied per group. The test was performed blind (test substances versus vehicle). Clobazam (8 mg/kg i.p.), administered 40 minutes before the test, was used as reference substance. Data with the test substances were analyzed by comparing treated groups with vehicle control using Kruskall-Wallis test followed by Mann-Whitney U tests. Data with the reference substance were analyzed using Mann-Whitney U tests.

Compound D dose-dependently reduced the number of marbles buried by wild type mice. FIG. 7 shows the mean±SEM number of marbles buried during a 30-minute test session. Groups (n=10) of mice were treated with a single i.p. dose of test drug at the indicated dose 20 minutes before the test session. Clobazam served as the positive control for the study and was administered i.p. 30 minutes before testing at a dose of 8 mg/kg. The effect of clobazam and 1 mg/kg Compound D were significantly different from that of vehicle (p<0.001, ANOVA, n=10).

### Example 3

### Characterization of Compounds

### Example 3.1

### Determination of the absolute configuration of (−)-4-methylbenzyl 3-fluoro-4-[(pyrimidin-2-ylamino)methyl]piperidine-1-carboxylate to be 3S,4R

A single crystal sample was crystallized by dissolving (−)-4-methylbenzyl 3-fluoro-4-[(pyrimidin-2-ylamino)methyl]piperidine-1-carboxylate in methanol (40 vol.) at 50° C. The sample was cooled to r.t., and the solvent was allowed to evaporate. A single crystal of prism morphology was isolated from the mother liquors with approximate dimensions 0.20×0.10×0.08 mm. This crystal was used for the structural determination.

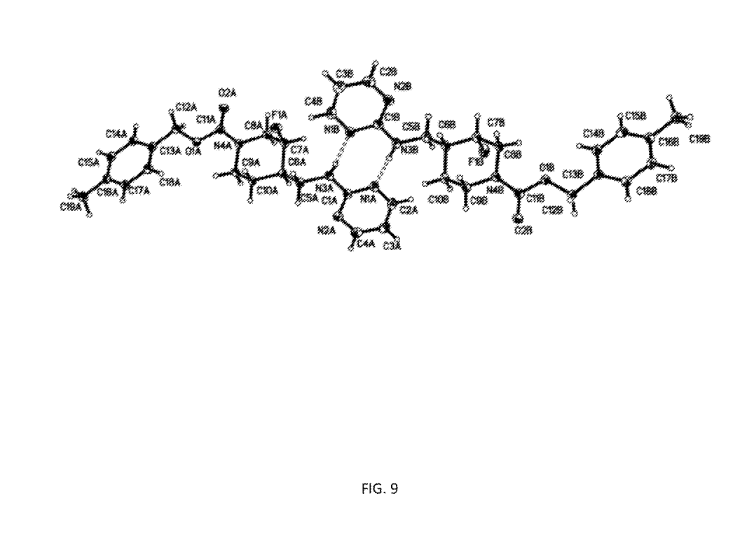

The absolute configuration of (−)-4-methylbenzyl 3-fluoro-4-[(pyrimidin-2-ylamino)methyl]-piperidine-1-carboxylate was determined to be 3S,4R. For the structure as presented in FIG. 9 with C6 in the R configuration and C7 in the S configuration, the Flack parameter=0.00(17). For the inverted structure with C6 in the S configuration and C7 in the R configuration, the Flack parameter=1.00(17). On the basis of the former result the absolute stereochemistry was assigned as depicted. Determination of the absolute structure using Bayesian statistics on Bijvoet differences (R. W. W. Hooft et al., J. Appl. Cryst., (2008), 41, 96-103), revealed that the probability of the absolute structure as presented being correct is 1.000, while the probabilities of the absolute structure being a racemic twin or false are both 0.000. The Flack equivalent and its uncertainty were calculated through this program to be 0.03(6). The calculation was based on 3086 Bijvoet pairs with a coverage of 86% (data not shown).

As shown in FIG. 9, the asymmetric unit contains two fully ordered molecules denoted as Molecule A and Molecule B. Anisotropic atomic displacement ellipsoids for the non-hydrogen atoms are shown at the 50% probability level. Hydrogen atoms are displayed with an arbitrarily small radius.

Table 3.1A lists the sample and crystal data for the single crystal of (−)-4-methylbenzyl 3-fluoro-4-[(pyrimidin-2-ylamino)methyl]piperidine-1-carboxylate that was characterized.

TABLE 3.1A

| Crystallization solvents | MeOH | |
|---|---|---|
| Crystallization method | Slow evaporation at RT | |
| Empirical formula | C₁₉H₂₃FN₄O₂ | |
| Formula weight | 358.41 | |
| Temperature | 100(2) K | |
| Wavelength | 1.54178 Å | |
| Crystal size | 0.200 × 0.100 × 0.080 mm | |
| Crystal habit | Colorless Prism | |
| Crystal system | Triclinic | |
| Space group | P1 | |
| Unit cell dimensions | a = 5.8583(6) Å | α = 100.445(12)° |
| | b = 10.2975(16) Å | β = 90.714(10)° |
| | c = 14.804(2) Å | γ = 92.827(11)° |

| | |
|---|---|
| Volume | 877.0(2) Å³ |
| Z | 2 |
| Density (calculated) | 1.357 Mg/m³ |
| Absorption coefficient | 0.803 mm⁻¹ |
| F(000) | 380 |

Table 3.1B lists the data collection and structure refinement information for the characterization of the single crystal of (−)-4-methylbenzyl 3-fluoro-4-[(pyrimidin-2-ylamino)methyl]piperidine-1-carboxylate.

TABLE 3.1B

| | |
|---|---|
| Diffractometer | SuperNova, Dual, Cu at zero, Atlas |
| Radiation source | SuperNova (Cu) X-ray Source, CuKα_{—} |
| Data collection method | omega scans |
| Theta range for data | 8.955 to 74.439° |
| collection | |
| Index ranges | −7 ≤ h ≤ 7, −12 ≤ k ≤ 12, −18 ≤ l ≤ 18 |
| Reflections collected | 15826 |
| Independent reflections | 6660 [R(int) = 0.0257] |
| Coverage of independent | 99.6% |
| reflections | |
| Variation in check | n/a |
| reflections | |
| Absorption correction | Semi-empirical from equivalents |
| Max. and min. transmission | 1.00000 and 0.95190 |
| Structure solution technique | Direct methods |
| Structure solution program | SHELXTL (Sheldrick, 2013) |
| Refinement technique | Full-matrix least-squares on F² |
| Refinement program | SHELXTL (Sheldrick, 2013) |
| Function minimized | Σ w(Fₒ² − F_{c}²)² |
| Data/restraints/parameters | 6660/3/480 |
| Goodness-of-fit on F² | 1.039 |
| Δ/σₘₐₓ | 0.000 |
| Final R indices | |
| 6252 data; I > 2σ (I) | R1 = 0.0342, wR2 = 0.0893 |
| all data | R1 = 0.0374, wR2 = 0.0947 |
| Weighting scheme | w = 1/[σ² (Fₒ²) + (0.0525P)² + 0.1752P] |
| | where P = (Fₒ² + 2Fc²)/3 |
| Absolute structure | 0.00(17) |
| parameter | |
| Extinction coefficient | n/a |
| Largest diff. peak and hole | 0.252 and −0.189 eÅ⁻³ |

Table 3.1C lists the atomic coordinates and equivalent isotropic atomic displacement parameters (Å²) for the single crystal of (−)-4-methylbenzyl 3-fluoro-4-[(pyrimidin-2-ylamino)methyl]-piperidine-1-carboxylate that was characterized.

TABLE 3.1C

| | x/a | y/b | z/c | U(eq)* |
|---|---|---|---|---|
| F1B | 0.4861(3) | 0.88539(15) | 0.79837(11) | 0.0303(3) |
| O1B | 0.7130(4) | 1.13874(19) | 1.03760(14) | 0.0204(4) |
| O2B | 1.0101(4) | 1.0178(2) | 1.06507(15) | 0.0252(5) |
| N1B | 0.3369(4) | 0.2533(2) | 0.77458(16) | 0.0216(5) |
| N2B | 0.0874(5) | 0.4223(2) | 0.84088(17) | 0.0241(5) |
| N3B | 0.4276(5) | 0.4718(2) | 0.77257(17) | 0.0231(5) |
| N4B | 0.6954(4) | 0.9212(2) | 0.98133(16) | 0.0206(5) |
| C1B | 0.2777(5) | 0.3809(3) | 0.79692(19) | 0.0205(6) |
| C2B | −0.0552(6) | 0.3272(3) | 0.8619(2) | 0.0255(6) |
| C3B | −0.0127(6) | 0.1943(3) | 0.8420(2) | 0.0273(7) |
| C4B | 0.1892(6) | 0.1627(3) | 0.7973(2) | 0.0242(6) |
| C5B | 0.3760(6) | 0.6095(3) | 0.7782(2) | 0.0233(6) |
| C6B | 0.4502(5) | 0.6963(3) | 0.86923(19) | 0.0203(6) |
| C10B | 0.7068(6) | 0.6973(3) | 0.8894(2) | 0.0235(6) |
| C9B | 0.7704(6) | 0.7874(3) | 0.9807(2) | 0.0231(6) |
| C8B | 0.4543(5) | 0.9309(3) | 0.9596(2) | 0.0216(6) |
| C7B | 0.3824(5) | 0.8374(3) | 0.8716(2) | 0.0207(6) |
| C11B | 0.8215(5) | 1.0246(3) | 1.03117(19) | 0.0192(6) |
| C12B | 0.8286(5) | 1.2513(3) | 1.0948(2) | 0.0218(6) |
| C13B | 0.6751(5) | 1.3659(3) | 1.10434(19) | 0.0195(6) |
| C14B | 0.4853(6) | 1.3676(3) | 1.0468(2) | 0.0225(6) |
| C15B | 0.3474(6) | 1.4749(3) | 1.0609(2) | 0.0235(6) |
| C16B | 0.3933(5) | 1.5828(3) | 1.1311(2) | 0.0214(6) |
| C17B | 0.5857(6) | 1.5801(3) | 1.1867(2) | 0.0228(6) |
| C18B | 0.7242(5) | 1.4738(3) | 1.1741(2) | 0.0214(6) |
| C19B | 0.2377(6) | 1.6964(3) | 1.1472(2) | 0.0266(7) |
| F1A | 0.2971(3) | −0.10330(16) | 0.59735(11) | 0.0277(3) |
| O1A | 0.4075(3) | −0.51069(18) | 0.37742(14) | 0.0199(4) |
| O2A | 0.1140(4) | −0.3889(2) | 0.34893(14) | 0.0233(5) |
| N1A | 0.7829(5) | 0.3810(2) | 0.63344(16) | 0.0223(5) |
| N2A | 1.0354(4) | 0.2123(2) | 0.56927(16) | 0.0223(5) |
| N3A | 0.6934(4) | 0.1620(2) | 0.63587(17) | 0.0212(5) |
| N4A | 0.4221(4) | −0.2944(2) | 0.43708(17) | 0.0199(5) |
| C1A | 0.8422(5) | 0.2538(3) | 0.61216(18) | 0.0188(6) |
| C2A | 0.9295(6) | 0.4711(3) | 0.6104(2) | 0.0253(7) |
| C3A | 1.1338(6) | 0.4410(3) | 0.5674(2) | 0.0270(7) |
| C4A | 1.1776(6) | 0.3081(3) | 0.5489(2) | 0.0267(7) |
| C5A | 0.7480(5) | 0.0255(3) | 0.63416(19) | 0.0205(6) |
| C6A | 0.6724(5) | −0.0675(3) | 0.54459(19) | 0.0179(5) |
| C7A | 0.4191(5) | −0.0664(3) | 0.5233(2) | 0.0210(6) |
| C8A | 0.3463(5) | −0.1612(3) | 0.4363(2) | 0.0209(6) |
| C9A | 0.6658(5) | −0.3031(3) | 0.4597(2) | 0.0221(6) |
| C10A | 0.7334(5) | −0.2093(3) | 0.5483(2) | 0.0199(6) |
| C11A | 0.2993(5) | −0.3966(3) | 0.38432(19) | 0.0188(6) |
| C12A | 0.2942(5) | −0.6224(3) | 0.3189(2) | 0.0210(6) |
| C13A | 0.4466(5) | −0.7368(3) | 0.30953(19) | 0.0196(6) |
| C14A | 0.3945(5) | −0.8462(3) | 0.24014(19) | 0.0207(6) |
| C15A | 0.5321(6) | −0.9540(3) | 0.2277(2) | 0.0223(6) |
| C16A | 0.7252(5) | −0.9553(3) | 0.2829(2) | 0.0214(6) |
| C17A | 0.7757(6) | −0.8473(3) | 0.3525(2) | 0.0224(6) |
| C18A | 0.6381(5) | −0.7387(3) | 0.3656(2) | 0.0215(6) |
| C19A | 0.8796(6) | −1.0701(3) | 0.2673(2) | 0.0275(7) |

*U(eq) is defined as one-third of the trace of the orthogonalized Uᵢⱼ tensor.

Table 3.1D lists the anisotropic atomic displacement parameters (Å²) for the single crystal of (−)-4-methylbenzyl 3-fluoro-4-[(pyrimidin-2-ylamino)methyl]piperidine-1-carboxylate that was characterized. (The anisotropic atomic displacement factor exponent takes the form:

−2π²[h²a*²U₁₁+ . . . +2hka*b*U₁₂].)

TABLE 3.1D

| | U₁₁ | U₂₂ | U₃₃ | U₂₃ | U₁₃ | U₁₂ |
|---|---|---|---|---|---|---|
| F1B | 0.0411(9) | 0.0244(7) | 0.0269(7) | 0.0086(6) | 0.0030(7) | 0.0017(6) |
| O1B | 0.0189(11) | 0.0179(9) | 0.0230(10) | −0.0005(8) | −0.0011(8) | 0.0022(8) |
| O2B | 0.0188(11) | 0.0250(10) | 0.0295(11) | −0.0018(8) | −0.0013(9) | 0.0031(9) |
| N1B | 0.0213(13) | 0.0204(11) | 0.0217(12) | −0.0002(9) | −0.0014(10) | 0.0025(9) |
| N2B | 0.0236(14) | 0.0263(12) | 0.0208(12) | −0.0011(9) | −0.0002(10) | 0.0052(10) |
| N3B | 0.0240(14) | 0.0169(11) | 0.0261(12) | −0.0024(9) | 0.0026(11) | 0.0032(10) |
| N4B | 0.0179(12) | 0.0186(11) | 0.0237(12) | −0.0006(9) | −0.0004(10) | 0.0038(9) |
| C1B | 0.0208(15) | 0.0221(13) | 0.0173(13) | 0.0000(10) | −0.0045(11) | 0.0021(11) |
| C2B | 0.0234(16) | 0.0329(16) | 0.0190(14) | 0.0012(11) | 0.0018(12) | 0.0018(12) |
| C3B | 0.0299(17) | 0.0301(16) | 0.0216(14) | 0.0055(12) | −0.0010(13) | −0.0028(13) |
| C4B | 0.0283(17) | 0.0220(13) | 0.0219(14) | 0.0034(11) | −0.0031(13) | 0.0018(12) |
| C5B | 0.0267(16) | 0.0205(14) | 0.0222(14) | 0.0026(11) | 0.0003(12) | 0.0010(12) |
| C6B | 0.0227(15) | 0.0167(13) | 0.0209(14) | 0.0011(10) | 0.0029(12) | 0.0028(11) |
| C10B | 0.0226(16) | 0.0190(13) | 0.0279(15) | 0.0005(11) | 0.0017(13) | 0.0043(11) |
| C9B | 0.0220(16) | 0.0175(13) | 0.0290(16) | 0.0015(11) | −0.0018(13) | 0.0044(12) |
| C8B | 0.0191(16) | 0.0182(13) | 0.0253(15) | −0.0033(11) | 0.0016(12) | 0.0051(11) |
| C7B | 0.0196(16) | 0.0191(13) | 0.0228(14) | 0.0021(10) | 0.0000(12) | 0.0019(11) |
| C11B | 0.0191(15) | 0.0198(13) | 0.0183(13) | 0.0021(10) | 0.0041(11) | 0.0023(11) |
| C12B | 0.0183(16) | 0.0191(14) | 0.0256(15) | −0.0019(11) | −0.0006(12) | −0.0012(12) |
| C13B | 0.0177(15) | 0.0204(14) | 0.0201(14) | 0.0032(11) | 0.0027(12) | −0.0004(11) |
| C14B | 0.0256(17) | 0.0191(14) | 0.0212(14) | 0.0000(11) | 0.0003(13) | −0.0005(12) |
| C15B | 0.0212(16) | 0.0271(15) | 0.0220(15) | 0.0051(12) | −0.0028(13) | −0.0026(12) |
| C16B | 0.0229(16) | 0.0198(14) | 0.0226(14) | 0.0062(11) | 0.0063(12) | 0.0016(12) |
| C17B | 0.0269(17) | 0.0185(13) | 0.0213(15) | −0.0001(11) | 0.0000(13) | −0.0018(12) |
| C18B | 0.0210(16) | 0.0212(13) | 0.0217(14) | 0.0033(11) | 0.0000(12) | −0.0009(11) |
| C19B | 0.0293(18) | 0.0219(15) | 0.0297(17) | 0.0066(12) | 0.0015(14) | 0.0047(13) |
| F1A | 0.0194(7) | 0.0346(8) | 0.0268(7) | −0.0007(6) | 0.0052(6) | 0.0008(6) |
| O1A | 0.0195(11) | 0.0159(9) | 0.0225(10) | −0.0011(7) | −0.0004(8) | 0.0006(8) |
| O2A | 0.0172(11) | 0.0239(10) | 0.0266(11) | −0.0017(8) | −0.0027(9) | 0.0031(8) |
| N1A | 0.0260(14) | 0.0191(11) | 0.0211(12) | 0.0020(9) | −0.0006(10) | 0.0017(10) |
| N2A | 0.0217(13) | 0.0250(12) | 0.0194(12) | 0.0019(9) | 0.0007(10) | 0.0019(10) |
| N3A | 0.0201(13) | 0.0181(11) | 0.0241(12) | −0.0004(9) | 0.0045(11) | 0.0024(10) |
| N4A | 0.0173(12) | 0.0163(11) | 0.0248(12) | −0.0005(9) | −0.0016(10) | 0.0026(9) |
| C1A | 0.0215(15) | 0.0187(13) | 0.0149(12) | −0.0005(10) | −0.0015(11) | 0.0015(11) |
| C2A | 0.0342(18) | 0.0194(13) | 0.0217(14) | 0.0032(11) | −0.0040(13) | −0.0007(12) |
| C3A | 0.0300(18) | 0.0285(16) | 0.0226(15) | 0.0074(12) | 0.0008(13) | −0.0072(13) |
| C4A | 0.0237(16) | 0.0349(16) | 0.0211(14) | 0.0048(12) | 0.0024(13) | −0.0006(13) |
| C5A | 0.0221(15) | 0.0173(13) | 0.0207(13) | −0.0001(10) | −0.0001(11) | 0.0013(11) |
| C6A | 0.0176(14) | 0.0164(13) | 0.0192(13) | 0.0027(10) | 0.0003(11) | −0.0011(10) |
| C7A | 0.0217(16) | 0.0172(13) | 0.0240(15) | 0.0025(11) | 0.0026(13) | 0.0041(11) |
| C8A | 0.0173(16) | 0.0214(13) | 0.0241(15) | 0.0038(11) | −0.0025(12) | 0.0035(11) |
| C9A | 0.0162(15) | 0.0199(13) | 0.0287(15) | 0.0003(11) | −0.0017(12) | 0.0040(11) |
| C10A | 0.0185(15) | 0.0146(12) | 0.0257(14) | 0.0012(10) | −0.0005(11) | 0.0024(11) |
| C11A | 0.0178(14) | 0.0202(13) | 0.0178(13) | 0.0016(10) | 0.0030(11) | 0.0017(11) |
| C12A | 0.0189(15) | 0.0188(14) | 0.0232(14) | −0.0006(11) | −0.0015(12) | −0.0010(11) |
| C13A | 0.0199(16) | 0.0191(14) | 0.0193(14) | 0.0027(11) | 0.0031(12) | −0.0012(11) |
| C14A | 0.0195(15) | 0.0215(14) | 0.0194(14) | 0.0004(11) | −0.0031(12) | −0.0026(12) |
| C15A | 0.0261(16) | 0.0195(13) | 0.0194(14) | −0.0006(10) | 0.0025(12) | −0.0023(12) |
| C16A | 0.0240(17) | 0.0169(13) | 0.0239(14) | 0.0051(11) | 0.0025(12) | 0.0017(12) |
| C17A | 0.0224(16) | 0.0224(14) | 0.0223(14) | 0.0039(11) | −0.0020(13) | 0.0021(12) |
| C18A | 0.0220(16) | 0.0219(14) | 0.0184(14) | −0.0019(11) | −0.0012(12) | 0.0005(12) |
| C19A | 0.0283(18) | 0.0226(15) | 0.0324(17) | 0.0054(12) | 0.0056(14) | 0.0055(13) |

What is claimed is:

1. A method for treating an anxiety disorder in an individual in need thereof, comprising administering to the individual an effective amount of an NR2B subunit-selective NMDA antagonist, wherein the antagonist is (+)-(1S,2S)-1-(4-hydroxyphenyl)-2-(4-hydroxy-4-phenyl-piperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoromethyl)phenyl-ethyl)piperidin-4-yl)-1H-pyrazolo-[3,4-d]pyrimidin-4-amine, or a pharmaceutically acceptable salt thereof.

2. The method of claim 1, wherein the anxiety disorder is obsessive compulsive disorder.

3. The method of claim 1, wherein the anxiety disorder is generalized anxiety disorder.

4. The method of claim 1, wherein the anxiety disorder is agoraphobia with panic disorder.

5. The method of claim 1, wherein the anxiety disorder is agoraphobia without panic disorder.

6. The method of claim 1, wherein the anxiety disorder is panic disorder.

7. The method of claim 1, wherein the anxiety disorder is post-traumatic stress disorder.

8. The method of claim 1, wherein the anxiety disorder is social anxiety disorder.

9. The method of any of claims 1-8, wherein the NR2B subunit-selective NMDA antagonist is (+)-(1S,2S)-1-(4-hydroxyphenyl)-2-(4-hydroxy-4-phenylpiperidino)-1-propanol: 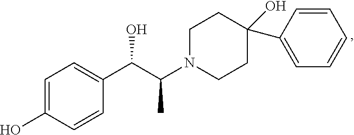 [SMILES: C[C@@H]([C@@H](O)c1ccc(O)cc1)N1CCC(O)(c2ccccc2)CC1]
or a pharmaceutically acceptable salt thereof.

10. The method of claim 9, wherein the anxiety disorder is obsessive compulsive disorder or generalized anxiety disorder.

11. The method of any of claims 1-8, wherein the NR2B subunit-selective NMDA antagonist is cis-4-methylbenzyl 3-fluoro-4-[(pyrimidin-2-ylamino)methyl]piperidine-1-carboxylate, or a pharmaceutically acceptable salt thereof.

12. The method of claim 11, wherein the NR2B subunit-selective NMDA antagonist is (−)-cis-4-methylbenzyl 3-fluoro-4-[(pyrimidin-2-ylamino)methyl]piperidine-1-carboxylate, or a pharmaceutically acceptable salt thereof.

13. The method of claim 11, wherein the NR2B subunit-selective NMDA antagonist is (+)-cis-4-methylbenzyl 3-fluoro-4-[(pyrimidin-2-ylamino)methyl]piperidine-1-carboxylate, or a pharmaceutically acceptable salt thereof.

14. The method of claim 11, wherein the NR2B subunit-selective NMDA antagonist is (3S,4R)-4-methylbenzyl 3-fluoro-4-[(pyrimidin-2-ylamino)methyl]piperidine-1-carboxylate: 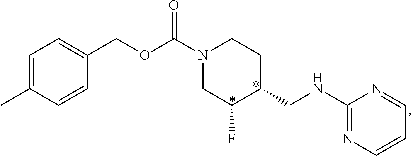 [SMILES: Cc1ccc(COC(=O)N2CC[C@H](CNc3ncccn3)[C@H](F)C2)cc1]
or a pharmaceutically acceptable salt thereof.

15. The method of claim 11, wherein the NR2B subunit-selective NMDA antagonist is (3R,4S)-4-methylbenzyl 3-fluoro-4-[(pyrimidin-2-ylamino)methyl]piperidine-1-carboxylate: 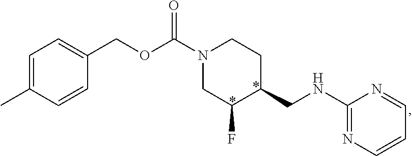 [SMILES: Cc1ccc(COC(=O)N2CC[C@@H](CNc3ncccn3)[C@@H](F)C2)cc1]
or a pharmaceutically acceptable salt thereof.

16. The method of any of claims 11-15, wherein the anxiety disorder is agoraphobia without panic disorder.

17. The method of any of claims 1-8, wherein the NR2B subunit-selective NMDA antagonist is N-(1-(2,2-difluoro-2-(4-trifluoromethyl)phenyl-ethyppiperidin-4-yl)-1H-pyrazolo[3,4-d]pyrimidin-4-amine, 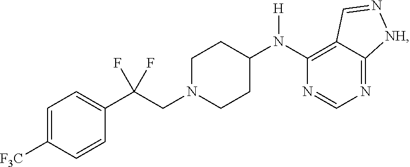 [SMILES: FC(F)(F)c1ccc(C(F)(F)CN2CCC(Nc3ncnc4[nH]ncc34)CC2)cc1]
or a pharmaceutically acceptable salt thereof.

18. The method of any of claims 1-18, wherein the NR2B subunit-selective NMDA antagonist is administered intravenously or intracranially of the individual in need thereof.

19. The method of any of claims 1-18, further comprising administering to the individual an effective amount of a therapeutic agent useful for treating an anxiety disorder.

20. The method of claim 19, wherein the therapeutic agent is a selective serotonin reuptake inhibitor, a tri-cyclic antidepressant, a benzodiazepine, an atypical antipsychotic or a serotonin-norepinephrine reuptake inhibitor.

21. The method of any one of claims 1-20, wherein the NR2B subunit-selective NMDA antagonist is administered in conjunction with behavioral therapy, electroconvulsive therapy, deep brain stimulation, or vagus nerve stimulation.

22. The method of any of claims 1-21, wherein the individual is a human.

23. A method for treating an autism spectrum disorder in an individual in need thereof comprising administering to the individual an effective amount of an NR2B subunit-selective NMDA antagonist, wherein the antagonist is (+)-(1S,2S)-1-(4-hydroxyphenyl)-2-(4-hydroxy-4-phenyl-piperidino)-1-propanol, cis-4-methylbenzyl 3-fluoro-4-[(pyrimidin-2-ylamino)methyl]piperidine-1-carboxylate, or N-(1-(2,2-difluoro-2-(4-trifluoromethyl)phenyl-ethyl)piperidin-4-yl)-1H-pyrazolo-[3,4-d]pyrimidin-4-amine, or a pharmaceutically acceptable salt thereof.

24. The method of claim 23, wherein the autism spectrum disorder is autism, Asperger's syndrome, or pervasive developmental disorder not otherwise specified (PDD-NOS).

25. The method of claim 23 or 24, wherein the NR2B subunit-selective NMDA antagonist is (+)-(1S,2S)-1-(4-hydroxyphenyl)-2-(4-hydroxy-4-phenylpiperidino)-1-propanol: 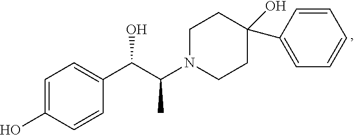 [SMILES: C[C@@H]([C@@H](O)c1ccc(O)cc1)N1CCC(O)(c2ccccc2)CC1]
or a pharmaceutically acceptable salt thereof.

26. The method of claim 23 or 24, wherein the NR2B subunit-selective NMDA antagonist is cis-4-methylbenzyl 3-fluoro-4-[(pyrimidin-2-ylamino)methyl]piperidine-1-carboxylate, or a pharmaceutically acceptable salt thereof.

27. The method of claim 26, wherein the NR2B subunit-selective NMDA antagonist is (−)-cis-4-methylbenzyl 3-fluoro-4-[(pyrimidin-2-ylamino)methyl]piperidine-1-carboxylate, or a pharmaceutically acceptable salt thereof.

28. The method of claim 26, wherein the NR2B subunit-selective NMDA antagonist is (+)-cis-4-methylbenzyl 3-fluoro-4-[(pyrimidin-2-ylamino)methyl]piperidine-1-carboxylate, or a pharmaceutically acceptable salt thereof.

29. The method of claim 26, wherein the NR2B subunit-selective NMDA antagonist is (3S,4R)-4-methylbenzyl 3-fluoro-4-[(pyrimidin-2-ylamino)methyl]piperidine-1-carboxylate: 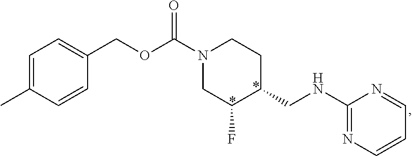 [SMILES: Cc1ccc(COC(=O)N2CC[C@H](CNc3ncccn3)[C@H](F)C2)cc1]
or a pharmaceutically acceptable salt thereof.

30. The method of claim 26, wherein the NR2B subunit-selective NMDA antagonist is (3R,4S)-4-methylbenzyl 3-fluoro-4-[(pyrimidin-2-ylamino)methyl]piperidine-1-carboxylate: 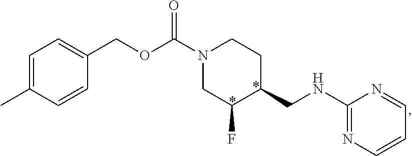 [SMILES: Cc1ccc(COC(=O)N2CC[C@@H](CNc3ncccn3)[C@@H](F)C2)cc1]
or a pharmaceutically acceptable salt thereof.

31. The method of claim 23 or 24, wherein the NR2B subunit-selective NMDA antagonist is N-(1-(2,2-difluoro-2-(4-trifluoromethyl)phenyl-ethyl)piperidin-4-yl)-1H-pyrazolo[3,4-d]pyrimidin-4-amine, 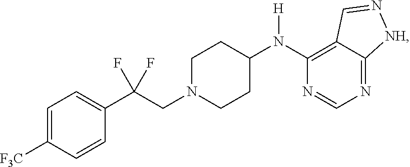 [SMILES: FC(F)(F)c1ccc(C(F)(F)CN2CCC(Nc3ncnc4[nH]ncc34)CC2)cc1]
or a pharmaceutically acceptable salt thereof.

32. The method of any of claims 23-31, wherein the NR2B subunit-selective NMDA antagonist is administered intravenously or intracranially of the individual in need thereof.

33. The method of any of claims 1-18, further comprising administering to the individual an effective amount of a therapeutic useful for treating an autism spectrum disorder.

34. The method of any of claims 23-33, further comprising administering to the individual an effective amount of a therapeutic agent useful for treating an autism spectrum disorder.

35. The method of claim 34, wherein the therapeutic agent is an atypical antipsychotic, a dopamine receptor agonist, a selective serotonin reuptake inhibitor, an atypical antipsychotic, a serotonin-norepinephrine reuptake inhibitor, a stimulant, secretin, oxytocin, or a typical antipsychotic.

36. The method of any one of claims 23-35, wherein the NR2B subunit-selective NMDA antagonist is administered in conjunction with behavioral therapy, electroconvulsive therapy, or hyperbaric oxygen therapy.

37. The method of any of claims 22-36, wherein the individual is a human.
